SEQUENCE LISTING (16 sequences)

SEQ ID NO: 1 - RNA, 23 nt, synthetic construct
  source 1..23: mol_type = other RNA
caaagtgcttacagtgcaggtag

SEQ ID NO: 2 - RNA, 23 nt, synthetic construct
  source 1..23: mol_type = other RNA
cccagtgttcagactacctgttc

SEQ ID NO: 3 - RNA, 22 nt, synthetic construct
  source 1..22: mol_type = other RNA
cactagattgtgagctcctgga

SEQ ID NO: 4 - RNA, 22 nt, synthetic construct
  source 1..22: mol_type = other RNA
tattgcacttgtcccggcctgt

SEQ ID NO: 5 - RNA, 21 nt, synthetic construct
  source 1..21: mol_type = other RNA
ctgacctatgaattgacagcc

SEQ ID NO: 6 - RNA, 22 nt, synthetic construct
  source 1..22: mol_type = other RNA
tgaggtagtaagttgtattgtt

SEQ ID NO: 7 - RNA, 22 nt, synthetic construct
  source 1..22: mol_type = other RNA
tgaggtagtagtttgtacagtt

SEQ ID NO: 8 - RNA, 22 nt, synthetic construct
  source 1..22: mol_type = other RNA
tgaggtagtagattgtatagtt

SEQ ID NO: 9 - RNA, 22 nt, synthetic construct
  source 1..22: mol_type = other RNA
tgaggtagtaggttgtatagtt

SEQ ID NO: 10 - RNA, 22 nt, synthetic construct
  source 1..22: mol_type = other RNA
tggagtgtgacaatggtgtttg

SEQ ID NO: 11 - RNA, 22 nt, synthetic construct
  source 1..22: mol_type = other RNA
agaggtagtaggttgcatagtt

SEQ ID NO: 12 - RNA, 22 nt, synthetic construct
  source 1..22: mol_type = other RNA
ttataaagcaatgagactgatt

SEQ ID NO: 13 - RNA, 22 nt, synthetic construct
  source 1..22: mol_type = other RNA
tgtaacagcaactccatgtgga

SEQ ID NO: 14 - RNA, 21 nt, synthetic construct
  source 1..21: mol_type = other RNA
ttcaagtaattcaggataggt

SEQ ID NO: 15 - RNA, 22 nt, synthetic construct
  source 1..22: mol_type = other RNA
aggtggtccgtggcgcgttcgc

SEQ ID NO: 16 - RNA, 21 nt, synthetic construct
  source 1..21: mol_type = other RNA
ctatatatcaaacatattcct

(19) United States
(12) Patent Application Publication
(10) Pub. No.: US 2025/0092460 A1
(43) Pub. Date: Mar. 20, 2025

(54) CIRCULATING MICRORNA SIGNATURES FOR PANCREATIC CANCER

(71) Applicants: Dana-Farber Cancer Institute, Inc., Boston, MA (US); Medical University of Lodz, Lodz (PL)

(72) Inventors: Dipanjan Chowdhury, Brookline, MA (US); Wojciech Fendler, Lodz (PL); Konrad Stawiski, Lodz (PL)

(21) Appl. No.: 18/577,387

(22) PCT Filed: Jul. 9, 2022
(86) PCT No.: PCT/US2022/036608

§ 371 Date: Jan. 9, 2024

Related U.S. Application Data
(60) Provisional application No. 63/220,195, filed on Jul. 9, 2021.

(51) Int. Cl. C12Q 1/6886; C12Q 1/6851

(52) U.S. Cl. CPC C12Q 1/6886; C12Q 1/6851; C12Q 2600/166; C12Q 2600/178

(57) ABSTRACT

Provided herein are methods and kits for measuring the amount of certain microRNA biomarkers in a sample collected from a subject. The microRNA biomarkers are associated with pancreatic cancer. Also provided are methods for screening a subject for the amount of certain microRNA biomarkers, as well as, methods for diagnosing, treating, or monitoring a subject suspected of having pancreatic cancer.

### RELATED APPLICATIONS

This application claims priority to U.S. Provisional Patent Application Ser. No. 63/220,195, filed Jul. 9, 2021. The entire disclosure of this application is hereby incorporated herein by reference in its entirety.

### STATEMENT REGARDING FEDERALLY SPONSORED RESEARCH OR DEVELOPMENT

### Background

Survival rates for pancreatic cancer depend on the size of the tumor and the degree of metastasis at the time of diagnosis. The earlier that pancreatic cancer is treated the better the prognosis. Unfortunately, pancreatic cancer usually shows few or no symptoms until it has advanced and spread. Up to 80% of cases are diagnosed at later stages, and are more difficult to treat (Pancreatic Cancer Prognosis, John Hopkins Medicine: Available at https://www.hopkinsmedicine.org/health/conditions-and-diseases/pancreatic-cancer/pancreatic-cancer-prognosis). In comparison to other cancers, the five-year survival rate for pancreatic cancer is very low at only 5 to 10% due to the high percentage of people who are diagnosed at stage IV when the cancer has metastasized.

MicroRNAs (miRNAs) are small regulatory RNA molecules that control gene expression by RNA silencing and post-transcriptional regulation. They are often tissue-specific and are dysregulated in many cancers. MicroRNAs have double-stranded hairpin structures and are more stable than messenger RNAs. Some miRNAs can be detected in the blood and the amounts remain stable in blood samples for years or even decades, providing a practical possibility for using them as biomarkers for noninvasive cancer diagnosis. However, most studies focus on miRNAs aberrantly expressed in tumor samples rather than blood samples.

There is a need in the art to identify circulating miRNAs for the purpose of accurate and robust diagnosis of early stage pancreatic cancer.

### SUMMARY

The instant disclosure provides methods for determining the presence or absence and/or the amount of microRNAs in a sample (e.g., blood sample) from a subject (e.g., human subject), as well as kits comprising probes to miRNAs. The instant disclosure also describes methods for treating a subject, as well as methods for screening blood samples of subjects for the presence or absence of certain miRNAs.

The disclosure provides, in one aspect, a method for diagnosing pancreatic cancer in a subject, the method comprising:
(a) obtaining a sample collected from the subject;
(b) detecting and quantifying one or more test microRNAs selected from the group consisting of hsa-miR-192-5p, hsa-miR-98-5p, hsa-let-7g-5p, hsa-let-7f-5p, hsa-let-7a-5p, hsa-miR-122-5p, hsa-let-7d-5p, hsa-miR-340-5p, hsa-miR-194-5p, hsa-miR-323a-5p, hsa-miR-190a-3p, and hsa-miR-26b-5p in the sample; and
(c) comparing the amounts of the test microRNAs determined in step (b) to a statistical model, thereby diagnosing pancreatic cancer in the subject.

In some embodiments, the method further comprises (d) detecting and quantifying one or more normalizing microRNAs selected from the group consisting of hsa-miR-17-5p, hsa-miR-199a-3p, hsa-miR-28-3p, and hsa-miR-92a-3p in the sample; and (e) normalizing the amounts of the test microRNAs using the amounts of the normalizing microRNAs quantified in step (d).

In some embodiments, methods are provided that comprise detecting and quantifying hsa-miR-192-5p, hsa-miR-98-5p, hsa-let-7g-5p, hsa-let-7f-5p, hsa-let-7a-5p, hsa-miR-122-5p, hsa-let-7d-5p, hsa-miR-340-5p, hsa-miR-194-5p, and hsa-miR-26b-5p.

In exemplary embodiments, methods are provided that comprise detecting and quantifying hsa-miR-192-5p, hsa-let-7g-5p, hsa-let-7a-5p, hsa-miR-194-5p, hsa-miR-122-5p, hsa-miR-340-5p, and hsa-miR-26b-5p. In some embodiments, methods are provided that consist of detecting and quantifying hsa-miR-192-5p, hsa-let-7g-5p, hsa-let-7a-5p, hsa-miR-194-5p, hsa-miR-122-5p, hsa-miR-340-5p, and hsa-miR-26b-5p.

In other embodiments, methods are provided that comprise detecting and quantifying hsa-miR-192-5p, hsa-miR-98-5p, hsa-let-7f-5p, hsa-let-7a-5p, hsa-miR-122-5p, hsa-let-7d-5p, hsa-miR-340-5p, and hsa-miR-194-5p.

In still other embodiments, methods are provided that comprise detecting and quantifying either hsa-miR-192-5p, hsa-miR-98-5p, hsa-let-7f-5p, hsa-let-7a-5p, hsa-miR-122-5p, hsa-let-7d-5p, hsa-miR-340-5p, hsa-miR-194-5p, and hsa-let-7g-5p; or hsa-miR-192-5p, hsa-miR-98-5p, hsa-let-7f-5p, hsa-let-7a-5p, hsa-miR-122-5p, hsa-let-7d-5p, hsa-miR-340-5p, hsa-miR-194-5p, and hsa-miR-26b-5p.

In some embodiments, methods are provided that comprise detecting and quantifying hsa-miR-323a-5p, hsa-miR-190a-3p, hsa-miR-192-5p, and hsa-let-7d-5p.

In other embodiments, methods are provided that comprise detecting and quantifying hsa-miR-192-5p and hsa-miR-194-5p.

In still other embodiments, methods are provided that comprise detecting and quantifying hsa-miR-192-5p, hsa-let-7a-5p, hsa-miR-194-5p, hsa-let-7f-5p, hsa-miR-122-5p, hsa-miR-340-5p, and hsa-miR-26b-5p.

In some embodiments, methods are provided that comprise detecting and quantifying at least 2, 3, 4, 5, 6, 7, 8, 9, or 10 test microRNAs.

In other embodiments, methods are provided that comprise detecting and quantifying reference microRNAs hsa-miR-17-5p, hsa-miR-199a-3p, hsa-miR-28-3p, and hsa-miR-92a-3p.

In further embodiments, methods are provided that comprise detecting and quantifying reference microRNAs hsa-miR-17-5p, hsa-miR-199a-3p, and hsa-miR-92a-3p.

In still other embodiments, methods are provided that comprise detecting and quantifying at least 2 or 3 normalizing microRNAs.

In other embodiments, methods are provided that comprise detecting and quantifying microRNAs by detecting binding of a sample to at least one probe capable of specifically hybridizing to each of the microRNAs or a cDNA thereof. In some embodiments, at least one of the probes comprises a detectable label. In other embodiments, each one of the probes comprises a detectable label.

In further embodiments, methods are provided that comprise detecting and quantifying microRNAs by a nucleic acid detection assay. In some embodiments, the assay is selected from the group consisting of microarray, RT-PCR, and RT-qPCR.

In additional embodiments, methods are provided that comprise detecting and quantifying microRNAs by reverse-transcribing the microRNA molecules in the sample, thereby obtaining a cDNA sample; and sequencing the cDNA sample. In some embodiments, the method further comprises amplifying the DNA molecules in the cDNA sample before sequencing the cDNA sample. In some embodiments, the method of detecting and quantifying microRNAs is performed using miRNA-seq.

In yet another aspect, the disclosure provides a kit comprising at least one test probe capable of specifically hybridizing to a microRNA selected from the group consisting of hsa-miR-192-5p, hsa-miR-98-5p, hsa-let-7g-5p, hsa-let-7f-5p, hsa-let-7a-5p, hsa-miR-122-5p, hsa-let-7d-5p, hsa-miR-340-5p, hsa-miR-194-5p, hsa-miR-323a-5p, hsa-miR-190a-3p, and hsa-miR-26b-5p, or a cDNA thereof.

In some embodiments, a kit is provided that comprises a test probe that specifically hybridizes to hsa-miR-192-5p, a test probe that specifically hybridizes to hsa-miR-98-5p, a test probe that specifically hybridizes to hsa-let-7g-5p, a test probe that specifically hybridizes to hsa-let-7f-5p, a test probe that specifically hybridizes to hsa-let-7a-5p, a test probe that specifically hybridizes to hsa-miR-122-5p, a test probe that specifically hybridizes to hsa-let-7d-5p, a test probe that specifically hybridizes to hsa-miR-340-5p, a test probe that specifically hybridizes to hsa-miR-194-5p, and a test probe that specifically hybridizes to hsa-miR-26b-5p, or a cDNA thereof.

In other embodiments, a kit is provided that comprises a test probe that specifically hybridizes to hsa-miR-192-5p, a test probe that specifically hybridizes to hsa-let-7g-5p, a test probe that specifically hybridizes to hsa-let-7a-5p, a test probe that specifically hybridizes to hsa-miR-122-5p, a test probe that specifically hybridizes to hsa-miR-340-5p, a test probe that specifically hybridizes to hsa-miR-194-5p, and
a test probe that specifically hybridizes to hsa-miR-26b-5p, or a cDNA thereof.

In some embodiments, a kit is provided that consists of a test probe that specifically hybridizes to hsa-miR-192-5p, a test probe that specifically hybridizes to hsa-let-7g-5p, a test probe that specifically hybridizes to hsa-let-7a-5p, a test probe that specifically hybridizes to hsa-miR-122-5p, a test probe that specifically hybridizes to hsa-miR-340-5p, a test probe that specifically hybridizes to hsa-miR-194-5p, and a test probe that specifically hybridizes to hsa-miR-26b-5p, or a cDNA thereof.

In additional embodiments, a kit is provided that comprises a test probe that specifically hybridizes to hsa-miR-192-5p, a test probe that specifically hybridizes to hsa-miR-98-5p, a test probe that specifically hybridizes to hsa-let-7f-5p, a test probe that specifically hybridizes to hsa-let-7a-5p, a test probe that specifically hybridizes to hsa-miR-122-5p, a test probe that specifically hybridizes to hsa-let-7d-5p, a test probe that specifically hybridizes to hsa-miR-340-5p, and a test probe that specifically hybridizes to hsa-miR-194-5p, or a cDNA thereof.

In further embodiments, a kit is provided that comprises a test probe that specifically hybridizes to hsa-let-7g-5p, or a test probe that specifically hybridizes to hsa-miR-26b-5p, or a cDNA thereof.

In some embodiments, a kit is provided that comprises a test probe that specifically hybridizes to hsa-miR-192-5p, and a test probe that specifically hybridizes to hsa-miR-194-5p, or a cDNA thereof.

In further embodiments, a kit is provided that comprises a test probe that specifically hybridizes to hsa-miR-192-5p, a test probe that specifically hybridizes to hsa-let-7a-5p, a test probe that specifically hybridizes to hsa-miR-194-5p, a test probe that specifically hybridizes to hsa-let-7f-5p, a test probe that specifically hybridizes to hsa-miR-122-5p, a test probe that specifically hybridizes to hsa-miR-340-5p, and a test probe that specifically hybridizes to hsa-miR-26b-5p, or a cDNA thereof.

In some embodiments, a kit is provided that comprises at least 2, 3, 4, 5, 6, 7, 8, 9, or 10 test probes.

In further embodiments, a kit is provided that comprises at least one normalizing probe capable of specifically hybridizing to a microRNA selected from the group consisting of hsa-miR-17-5p, hsa-miR-199a-3p, hsa-miR-28-3p, and hsa-miR-92a-3p, or a cDNA thereof.

In some embodiments, a kit is provided that comprises a normalizing probe capable of specifically hybridizing to hsa-miR-17-5p, a normalizing probe capable of specifically hybridizing to hsa-miR-199a-3p, a normalizing probe capable of specifically hybridizing to hsa-miR-28-3p, and a normalizing probe capable of specifically hybridizing to hsa-miR-92a-3p, or a cDNA thereof.

In additional embodiments, a kit is provided that comprises a normalizing probe capable of specifically hybridizing to hsa-miR-17-5p, a normalizing probe capable of specifically hybridizing to hsa-miR-199a-3p, and a normalizing probe capable of specifically hybridizing to hsa-miR-92a-3p, or a cDNA thereof.

In other embodiments, a kit is provided that comprises at least 2 or 3 normalizing probes.

In additional embodiments, a kit is provided that comprises no normalizing probes.

In further embodiments, a kit is provided that comprises at least one probe that comprises a detectable label. In some embodiments, each one of the probes comprises a detectable label.

In some embodiments, a kit is provided that comprises a reagent for reverse transcription of a microRNA molecule.

In still another aspect, the disclosure provides a method for treating a subject suspected of having a pancreatic cancer, the method comprising:
(a) obtaining a sample collected from the subject;
(b) detecting and quantifying one or more test microRNAs selected from the group consisting of hsa-miR-192-5p, hsa-miR-98-5p, hsa-let-7g-5p, hsa-let-7f-5p, hsa-let-7a-5p, hsa-miR-122-5p, hsa-let-7d-5p, hsa-miR-340-5p, hsa-miR-194-5p, hsa-miR-323a-5p, hsa-miR-190a-3p, and hsa-miR-26b-5p in the sample;
(c) comparing the amounts of the test microRNAs determined in step (b) to a statistical model; and
(d) selecting a subject for more invasive testing and/or surveillance of pancreatic cancer based on the comparison of step (c); and optionally administering treatment to the subject for pancreatic cancer.

In some embodiments, the more invasive testing is selected from the group consisting of magnetic resonance imaging (MRI), computed tomography (CT) scan, x-ray, positron emission tomography and computed tomography (PET-CT) scan, endoscopy, ultrasound, nuclear scan, and biopsy.

In other embodiments, the surveillance of pancreatic cancer comprises periodic image testing selected from the group consisting of magnetic resonance imaging (MRI), computed tomography (CT) scan, x-ray, positron emission tomography and computed tomography (PET-CT) scan, endoscopy, ultrasound, and nuclear scan. In some embodiments, the periodic imaging occurs every 3, 6, or 12 months.

In still other embodiments, the subject is administered treatment for pancreatic cancer. In some embodiments, the treatment administered is selected from the group consisting of surgery, chemotherapy, immunotherapy, and radiation therapy. In some embodiments, the treatment comprises the immunotherapy pembrolizumab. In some embodiments, the treatment comprises surgery. In some embodiments, the treatment comprises chemotherapy and immunotherapy. In some embodiments, the treatment comprises chemotherapy and radiation.

In additional embodiments, the subject is administered chemotherapy treatment for pancreatic cancer. In some embodiments, the chemotherapy is selected from the group consisting of a taxane, an antimetabolite drug, a platinum chemotherapy, an alkylating agent, an agent that inhibits DNA replication, a PARP inhibitor, and an antineoplastic chemotherapy. In some embodiments, the chemotherapy comprises a taxane selected from the group consisting of paclitaxel, docetaxel, and albumin-bound paclitaxel. In some embodiments, the chemotherapy comprises an antimetabolite drug selected from the group consisting of gemcitabine hydrochloride, 5-fluorouracil (5-FU), and capecitabine. In some embodiments, the chemotherapy comprises the platinum chemotherapy oxaliplatin. In some embodiments, the chemotherapy comprises the alkylating agent cisplatin. In some embodiments, the chemotherapy comprises an agent that inhibits DNA replication selected from the group consisting of irinotecan and liposomal irinotecan. In some embodiments, the chemotherapy comprises the PARP inhibitor olaparib. In some embodiments, the chemotherapy comprises an antineoplastic chemotherapy selected from the group consisting of everolimus, erlotinib hydrochloride, sunitinib, and mitomycin.

In further embodiments, the subject is administered a drug combination treatment for pancreatic cancer. In some embodiments, the drug combination is selected from the group consisting of FOLFIRINOX (Folinic Acid, Fluorouracil, Irinotecan Hydrochloride, and Oxaliplatin), GEMCITABINE-CISPLATIN (Gemcitabine Hydrochloride and Cisplatin), GEMCITABINE-OXALIPLATIN (Gemcitabine Hydrochloride and Oxaliplatin), and OFF (Oxaliplatin, Fluorouracil, and Folinic Acid).

In an additional aspect, the disclosure provides an analytical method for diagnosing pancreatic cancer in a subject, comprising the steps of:
a) detecting and quantifying one or more test microRNAs selected from the group consisting of hsa-miR-192-5p, hsa-miR-98-5p, hsa-let-7g-5p, hsa-let-7f-5p, hsa-let-7a-5p, hsa-miR-122-5p, hsa-let-7d-5p, hsa-miR-340-5p, hsa-miR-194-5p, hsa-miR-323a-5p, hsa-miR-190a-3p, and hsa-miR-26b-5p in the sample from said subject;
b) analyzing the amount of the one or more test microRNAs quantified in step a) in a neural network to determine the probability that the subject has pancreatic cancer; and
c) assigning the subject as probable to have pancreatic cancer based on the analysis of step b).

In some embodiments, the assignment of the subject as probable to have pancreatic cancer has an accuracy rate of greater than 50%, 60%, 70%, 80%, or 90%. In some embodiments, the assignment of the subject as probable to have pancreatic cancer has a specificity rate of greater than 50%, 60%, 70%, 80%, or 90%. In some embodiments, the assignment of the subject as probable to have pancreatic cancer has a sensitivity rate of greater than 50%, 60%, 70%, 80%, or 90%.

In further embodiments, the method comprises obtaining a sample. In some embodiments, the sample is a blood sample or a pancreatic sample. In some embodiments, the blood sample is selected from the group consisting of plasma, serum, and whole blood.

In yet other embodiments, the method comprises obtaining a sample collected from the subject. In some embodiments, the subject is a human subject. In some embodiments, the subject is at a higher risk of developing pancreatic cancer. In some embodiments, the subject has diabetes. In some embodiments, the subject has pancreatitis. In some embodiments, has a family history of pancreatic cancer or pancreatitis. In some embodiments, the subject is at a higher risk for developing pancreatic cancer due to a genetic mutation. In some embodiments, the subject has a mutation in a gene selected from the group consisting of BRCA1, BRCA2, PALB2, TP53, MLH1, CDKN2A, and ATM.

In further embodiments, the method comprises the use of a statistical model. In some embodiments, the statistical model comprises one or more models selected from the group consisting of linear discriminant analysis, logistic regression, multivariate adaptive regression splines, naive Bayes, neural network, support vector machine, decision tree, K nearest neighbors, functional tree, least absolute deviation (LAD) tree, Bayesian network, elastic net regression, and random forest.

### BRIEF DESCRIPTION OF THE DRAWINGS

FIG. 1 depicts a schematic of the study design of Example 1 for producing a circulating miRNA signature from human sera using two independent cohorts of patients.

The schematic also depicts how the patients were assigned to a training set, a test set, and a validation set. The schematic also depicts the steps of using a series of statistical tools to create an algorithm, creating a final set of miRNAs, calibrating the model, and validation of the model using qPCR.

FIG. 2A-B depict a variable selection study of the training set of Example 1. Ten miRNAs were selected as having a family-wise error rate (FWER) p value <0.05 (with a Bonferroni-adjusted p value). The volcano plot (FIG. 2A) and table of results (FIG. 2B) illustrate that for these 10 miRNAs, 3 were upregulated and 7 were downregulated.

FIG. 3A-C depict the development of a classification model in Example 1 using logistic regression performance further refined with backward stepwise logistic regression.

FIG. 3A depicts a plot of specificity versus sensitivity for the miRNA models tested. FIG. 3B depicts the calculated values for the 4 miRNAs used in the final model. These results show that the model fits the training and test set very well with a Hosmer Lemeshow value=4.4927 and a p value=0.810161. FIG. 3C depicts the sensitivity and specificity in detecting cancer in cancer positive samples versus control. The final logistic regression model of 4 miRNAs showed a sensitivity of 79.3% and a specificity of 84.1%.

FIG. 4A-B depict the development of a classification model in Example 1 using artificial neural network with sensitivity analysis used to reduce the number of miRNAs in the analysis. FIG. 4A depicts a plot of specificity versus sensitivity for the model miRNA models tested. The artificial neural network requires the following 8 miRNAs: hsa-miR-192-5p, hsa-let-7a-5p, hsa-let-7d-5p, hsa-miR-194-5p, hsa-miR-98-5p, hsa-let-7f-5p, hsa-miR-122-5p, and hsa-miR-340-5p. FIG. 4B depicts sensitivity and specificity in detecting cancer in cancer positive samples versus control. The final artificial neural network showed a sensitivity of 71.4% and a specificity of 90.9%.

FIG. 5A-B depicts the splitting of the dataset from Example 1 into a training set, testing set, and validation set in order to create diagnostic models using logistic regression, artificial neural network on the raw dataset, and artificial neural network on the SMOTE balanced dataset. As shown in FIG. 5A, the dataset of the two groups of Polish samples was split for modeling. FIG. 5B depicts the use of the SMOTE technique to create a balanced dataset from the training set.

FIG. 6A-B illustrate the use in Example 1 of logistic regression of a two miRNA (hsa-miR-192-5p and hsa-miR-194-5p) model to evaluate the performance of both the test and validation dataset. FIG. 6B illustrates that this model has a rate of 66% sensitivity and 74% specificity in predicting cancer in samples with observed cancer versus control.

FIG. 7A-B depict the results of the neural network model of classic (non-SMOTE modified) data in Example 1. These results were generated using the clinical data including age and sex, as well as, all 10 miRNAs. The following miRNAs were used for normalization: hsa-miR-17-5p, hsa-miR-92a-3p, and hsa-miR-199a-3p. FIG. 7A shows the area under the ROC curve (AUC) for the training set to be 0.8475. FIG. 7B provides values of 82.57% accuracy, 59.72% sensitivity, and 93.84% specificity for the training and test datasets. FIG. 7B provides values of 83.02% for accuracy, 64.71% for sensitivity, and 91.67% for specificity for the validation data set.

FIG. 8A-B depict the results of the neural network model on the SMOTE balanced dataset in Example 1. These results were generated using the clinical data set and a trimmed miRNA set comprising hsa-miR-192-5p, hsa-let-7a-5p, hsa-miR-194-5p, hsa-let-7f-5p, hsa-miR-122-5p, hsa-miR-340-5p, and hsa-miR-26b-5p. The following miRNAs were used for normalization: hsa-miR-17-5p, hsa-miR-92a-3p, and hsa-miR-199a-3p. FIG. 8A shows the AUC for the data set to be 0.8971. FIG. 8B provides values of 84.86% accuracy, 79.17% sensitivity, and 87.67% specificity for the training and test datasets. FIG. 8B provides values of 86.79% accuracy, 76.47% sensitivity, and 91.67% specificity for the validation dataset.

### DETAILED DESCRIPTION

The present disclosure provides methods and kits for measuring the amount of certain miRNA biomarkers in a sample collected from a subject. The miRNA biomarkers are associated with cancer (e.g., pancreatic cancer). Specifically, unique combinations of test and normalizing miRNAs are described that are used to predict, in a statistically relevant manner, an increased probability that a subject has cancer (e.g., pancreatic cancer). The use of these miRNA combinations provides a non-invasive cancer detection method that is useful for monitoring an individual's susceptibility to disease. The detection method may be used either alone or in combination with other known diagnostic methods. The methods described herein are particularly useful for detecting or diagnosing pancreatic cancer. For example, these methods are effective in distinguishing between pancreatic cancer and pancreatitis, the most common differential diagnosis. Also provided herein are methods of surveilling and treating subjects diagnosed with cancer (e.g., pancreatic cancer).

It is to be understood that the methods described in this disclosure are not limited to particular methods and experimental conditions disclosed herein, as such methods and conditions may vary. It is also to be understood that the terminology used herein is for the purpose of describing particular embodiments only, and is not intended to be limiting.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by MR Green and J. Sambrook and Harlow et al., Antibodies: A Laboratory Manual, Chapter 14, Cold Spring Harbor Laboratory, Cold Spring Harbor (2013, 2^{nd} edition).

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclatures used in connection with cell and tissue culture, molecular biology, immunology, microbiology, genetics and protein and nucleic acid chemistry and hybridization described herein are those well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

### Methods for Detection of RNA and DNA

The present disclosure provides compositions and methods for the diagnosis and treatment of cancer. In an exemplary embodiment, provided herein is a diagnostic test for pancreatic cancer that is both sensitive and specific. This diagnostic test relies on the detection and quantification of nucleic acids. Specifically, a diagnostic test is provided that relies on the detection and quantification of microRNAs.

Provided herein are methods for the quantification and detection of nucleic acids. As used herein, the term “nucleic acid” refers to a polymer of two or more nucleotides or nucleotide analogues (such as ribonucleic acid having methylene bridge between the 2′-O and 4′-C atoms of the ribose ring) capable of hybridizing to a complementary nucleic acid. As used herein, this term includes, without limitation, DNA, RNA, LNA, and PNA.

Specifically, provided herein are methods for the detection and quantification of microRNAs. The term “microRNAs” or “miRNAs” as used herein, refers to small noncoding ribonucleic acid (RNA) gene products between 19 and 26 nucleotides long that form a hairpin secondary structure. MicroRNAs described herein are named using the nomenclature set forth in Ambros et al., RNA. 2003 March; 9(3):277-9, incorporated herein by reference, and sequences may be found at mirbase.org.

In some embodiments, detection and quantification of the miRNAs includes the use of a test microRNA. As used herein, the term “test microRNA” refers to a microRNA the presence or absence and/or amount of which is determined, for example, for diagnosis purpose (e.g., using an algorithm). In some embodiments, the presence or absence and/or amount of one or more test microRNAs can be used additionally for normalization purpose.

The phrase “detecting and quantifying one or more test microRNAs”, as used herein, encompasses any method that may be used to measure the concentration, absolute value or presence of a microRNA. Exemplary methods for determining the amounts of microRNAs include sequencing (e.g., Gilbert sequencing, Sanger sequencing, SMRT sequencing or next-generation sequencing), microarray detection, PCR, RT-PCR, real-time qPCR, and real-time RT-qPCR.

In some embodiments, detection and quantification of the miRNAs is performed using normalization. As used herein, the term “normalize” or “normalizing” refers to adjusting a first measured value (e.g., level of a gene of interest) relative to a second measured value (e.g., level of a housekeeping gene), wherein the first and second measured values are measured from the same sample (e.g., different portions of the same homogenous sample), and wherein the second measured value is correlated to the quantity and/or quality of the sample. Normalization allows obtaining a relative amount of the first value that is not affected by the quantity and/or quality of the sample that may vary from individual sample preparation. As used herein, the term “normalizing microRNA” or “reference microRNA” refers to a microRNA that is known to have a stable amount in a sample (e.g., a blood sample) and is used to normalize the measured value of a test microRNA in the sample. A single normalizing microRNA may be used to normalize the measured amount of a target microRNA in a sample, or an averaged value of multiple microRNAs may be used for normalization. In certain embodiments, normalization may be calculated by: Number of amplification cycles (average of the normalizer microRNA)−number of amplification cycles (miR of interest).

In exemplary embodiments, one or more, or various combinations of, the miRNAs of Tables 1 and 2 will be quantified using the methods disclosed herein. Table 1 provides reference miRNAs for normalizing results. Table 2 provides test miRNAs that serve as biomarkers for cancer (e.g., pancreatic cancer).

TABLE 1
Reference miRNAs for Normalizing Results

| SEQ | MicroRNA | |
|---|---|---|
| ID NO: | miRBase ID | Sequence |
| 1 | hsa-miR-17-5p | CAAAGUGCUUACAGUGCAGGUAG |
| 2 | hsa-miR-199a-3p | CCCAGUGUUCAGACUACCUGUUC |
| 3 | hsa-miR-28-3p | CACUAGAUUGUGAGCUCCUGGA |
| 4 | hsa-miR-92a-3p | UAUUGCACUUGUCCCGGCCUGU |

TABLE 2
Test miRNAs for Diagnosing Pancreatic Cancer

| SEQ | MicroRNA | |
|---|---|---|
| ID NO: | miRBase ID | Sequence |
| 5 | hsa-miR-192-5p | CUGACCUAUGAAUUGACAGCC |
| 6 | hsa-miR-98-5p | UGAGGUAGUAAGUUGUAUUGUU |
| 7 | hsa-let-7g-5p | UGAGGUAGUAGUUUGUACAGUU |
| 8 | hsa-let-7f-5p | UGAGGUAGUAGAUUGUAUAGUU |
| 9 | hsa-let-7a-5p | UGAGGUAGUAGGUUGUAUAGUU |
| 10 | hsa-miR-122-5p | UGGAGUGUGACAAUGGUGUUUG |
| 11 | hsa-let-7d-5p | AGAGGUAGUAGGUUGCAUAGUU |
| 12 | hsa-miR-340-5p | UUAUAAAGCAAUGAGACUGAUU |
| 13 | hsa-miR-194-5p | UGUAACAGCAACUCCAUGUGGA |
| 14 | hsa-miR-26b-5p | UUCAAGUAAUUCAGGAUAGGU |
| 15 | hsa-miR-323a-5p | AGGUGGUCCGUGGCGCGUUCGC |
| 16 | hsa-miR-190a-3p | CUAUAUAUCAAACAUAUUCCU |

In some embodiments, the levels of 1, 2, 3, 4, 5, 6, 7, 8, 9, 10, 11, or 12 of the test miRNAs in Table 2 are detected and quantified. In an exemplary embodiment, the levels of 7 of the test miRNAs in Table 2 are detected and quantified.

In some embodiments, the levels of 1, 2, 3, or 4 of the reference miRNAs in Table 1 are detected and quantified. In an exemplary embodiment, the levels of all 4 reference miRNAs in Table 1 are detected and quantified. In an additional exemplary embodiment, the levels of 4 reference miRNAs in Table 1 and 7 of the test miRNAs in Table 2 are detected and quantified.

In exemplary embodiments, the miRNAs to be detected and quantified are obtained from a sample from a subject. The term “subject,” as used herein, refers to a mammal, e.g., a human, a domestic animal or livestock including a cat, a dog, cattle or a horse. As used herein, the term “sample” refers to a biological specimen of material derived from a subject, such as a tissue or fluid.

As used herein, “obtaining a sample collected from a subject” encompasses any suitable means disclosed herein, as well as, routine clinical methods for retrieving a biological specimen from a subject. Samples can be directly taken from a subject or can be obtained from a third party. Sample collection can be performed by, for example, a health care provider, such as a physician, physician assistant, nurse, veterinarian, dentist, chiropractor, paramedic, dermatologist, oncologist, gastroenterologist, or surgeon. Samples include, but are not limited to, blood, mucosa (e.g., saliva), lymph, urine, stool, and solid tissue samples. In an exemplary embodiment, fluid samples are collected from a subject. Procedures for obtaining fluid samples from a subject are well known, including the procedure for collecting and processing whole blood and lymph.

In exemplary embodiments, the sample obtained from a subject is a blood sample. As used herein, the term “blood sample” refers to an amount of blood taken from a subject, such as whole blood, or a component portion of blood taken from a subject, such as plasma, which lacks cells normally contained in whole blood (e.g., erythrocytes, leukocytes, and platelets), or serum which is plasma that lacks fibrinogen and some clotting factors. In some embodiments, the sample is a tissue sample. A tissue sample can be obtained by biopsy. In some embodiments, the sample is a tissue biopsy (e.g. needle biopsy, CT-guided needle biopsy, aspiration biopsy, endoscopic biopsy, bronchoscope biopsy, bronchial lavage, incision biopsy, resection biopsy, punch biopsy, slice biopsy, skin biopsy, bone marrow biopsy, or electrochemical loop resection).

In some embodiments, detection and quantification of the miRNAs includes a step or steps of binding between nucleic acids. As used herein, the term “bind” or “binding” refers to non-covalent or covalent interaction between two molecules, such as between two complementary nucleic acids.

In some embodiments, detection and quantification of the miRNAs includes a step or steps of hybridization between nucleic acids. The term “hybridize” as used herein, refers to annealing of a first single-stranded nucleic acid to a second complementary single-stranded nucleic, in which complementary nucleotides of the first and second nucleic acids pair by hydrogen bonding. As used herein, the term “specifically hybridizing” refers to non-covalent interaction between a first nucleic acid molecule (e.g., a nucleic acid probe having a certain nucleotide sequence) and a second nucleic acid molecule (e.g., a microRNA having a nucleotide sequence complementary to that of the nucleic acid probe). Hybridization conditions have been described in the art and are known to one of skill in the art. In some embodiments, the condition for detecting the hybridization is a suitable condition of a nucleic acid detection assay (e.g., microarray, RT-PCR, or RT-qPCR). The likelihood of hybridization between two nucleic acids correlates with the nucleotide sequence complementary between the two nucleic acids. An oligonucleotide “specifically hybridizes” to a target polynucleotide if the oligonucleotide hybridizes to the target under physiological conditions, with a Tm greater than 37° C., greater than 45° C., preferably at least 50° C., and typically 60° C.-80° C. or higher. The “Tm” of an oligonucleotide is the temperature at which 50% hybridizes to a complementary polynucleotide. Tm is determined under standard conditions in physiological saline, as described, for example, in Miyada et al., Methods Enzymol. 154:94-107 (1987).

In some embodiments, detection and quantification of the miRNAs includes a step of interaction of complementary nucleic acids. Polynucleotides are described as “complementary” to one another when hybridization occurs in an antiparallel configuration between two single-stranded polynucleotides. Complementarity (the degree that one polynucleotide is complementary with another) is quantifiable in terms of the proportion of bases in opposing strands that are expected to form hydrogen bonds with each other, according to generally accepted base-pairing rules.

In some embodiments, detection and quantification of the miRNAs is performed by PCR. The term “PCR,” as used herein, refers to polymerase chain reaction for amplifying an amount of target DNA. PCR relies on thermal cycling, which consists of cycles of repeated heating and cooling of a reaction for DNA denaturation, annealing and enzymatic elongation of the amplified DNA. First, the strands of the DNA are separated at a high temperature in a process called DNA melting or denaturing. Next, the temperature is lowered, allowing the primers and the strands of target DNA to selectively bind or anneal, creating templates for DNA polymerase to amplify the target DNA. Next, at a working temperature of the DNA polymerase, template-dependent DNA synthesis occurs. These steps are repeated to create many copies of the target DNA.

In some embodiments, detection and quantification of the miRNAs includes the use of a primer. A “primer,” as used herein, refers to a short, single-stranded DNA sequence that selectively binds to a target DNA sequence and enables addition of new deoxyribonucleotides by DNA polymerase at the 3′ end. According to certain embodiments, the forward primer is 18-35, 19-32 or 21-31 nucleotides in length. The nucleotide sequence of the forward primer is not limited, so long as it specifically hybridizes with part of or an entire target site, and its Tm value may be within a range of 50° C. to 72° C., in particular may be within a range of 58° C. to 61° C., and may be within a range of 59° C. to 60° C. The nucleotide sequence of the primer may be manually designed to confirm the Tm value using a primer Tm prediction tool. Primer nucleotides may include nucleotide analogues and/or modified nucleotides, such as LNA or PNA.

In some embodiments, detection and quantification of the miRNAs is performed using RT-PCR. As used herein, the term “RT-PCR” refers to reverse transcription polymerase chain reaction, a process for amplifying RNA. RNA molecules are reverse transcribed to complementary DNA (cDNA) using reverse transcriptase and then using PCR to amplify the resulting cDNA.

In some embodiments, detection and quantification of the miRNAs is performed using RT-qPCR. As used herein, the term “RT-qPCR” refers to reverse transcription quantitative polymerase chain reaction, a variant of RT-PCR in which amplification of cDNA during the RT-PCR process is quantitatively detected in real time using a probe that detects amplified target DNA. For example, in some embodiments, self-quenching nucleic acid probes are added to the reaction mixture. The self-quenching nucleic acid probes only fluoresce when they bind a target sequence. As each cycle of PCR is completed, the self-quenching probes bind to the amplified DNA, unquench and fluoresce with exposure to a light excitation source. As DNA is amplified, increased probe and target binding results in increased fluorescence of the self-quenching nucleic acid probe. Detection of the fluorescing probes after each amplification cycle allows real-time measurement of the amplification process, as increasing amounts of the nucleic acid probe bind with amplified target DNA and fluoresce. In some embodiments, an intercalating dye probe is added to the reaction mixture that fluoresces upon interaction with double-stranded nucleic acids. The increase in dye fluorescence during the amplification process allows the measurement of DNA amplification in real-time, as increasing amounts of the dye probe intercalate with the increasing amounts of target DNA being amplified.

In some embodiments, detection and quantification of the miRNAs involves the use of a probe. As used herein, the term “probe” refers to a molecule or complex that is used to determine the presence or absence and/or amount of a microRNA in a sample (e.g., a blood sample). In certain embodiments, the probe comprises a nucleic acid moiety (e.g., DNA, modified DNA, or modified RNA) that is capable of specifically hybridizing to the microRNA or a complementary DNA (cDNA) thereof. In certain embodiments, the probe comprises a sequence of at least 15, 16, 17, 18, 19, 20, 21, 22, 23, 24, or 25 contiguous nucleotides identical or complementary to the microRNA. In certain embodiments, a probe further comprises a detectable label that is conjugated, covalently or non-covalently, to the nucleic acid moiety. Exemplary detectable labels include without limitation a fluorophore, a small molecule (e.g., a small molecule of the avidin family), an enzyme, an antibody or antibody fragment, or a nucleic acid sequence not present in the subject in a form that is linked to the microRNA (e.g., a barcode sequence). Accordingly, the probe may be a fluorophore-labeled nucleic acid having a nucleotide sequence that is complementary to a nucleotide sequence of a microRNA.

In some embodiments, detection and quantification of the miRNAs includes the use of a normalizing probe. As used herein, the term “normalizing probe” refers to a probe that is used to determine the presence or absence and/or amount of a normalizing microRNA in a sample (e.g., a blood sample). In certain embodiments, the normalizing probe comprises a nucleic acid moiety (e.g., DNA, modified DNA, or modified RNA) that is capable of specifically hybridizing to a normalizing microRNA or a complementary DNA (cDNA) thereof.

In some embodiments, detection and quantification of the miRNAs includes the use of a test probe. As used herein, the term “test probe” refers to a probe that is used to determine the presence or absence and/or amount of a test microRNA in a sample (e.g., a blood sample). In certain embodiments, the test probe comprises a nucleic acid moiety (e.g., DNA, modified DNA, or modified RNA) that is capable of specifically hybridizing to a test microRNA or a complementary DNA (cDNA) thereof.

In some embodiments, detection and quantification of the miRNAs includes the use of a reagent for amplification of a DNA sequence. The phrase “a reagent for amplification of a DNA sequence” includes, but is not limited to: (1) a heat-stable DNA polymerase; (2) deoxynucleotide triphosphates (dNTPs); (3) a buffer solution, providing a suitable chemical environment for optimum activity, binding kinetics, and stability of the DNA polymerase; (4) bivalent cations such as magnesium or manganese ions; and (5) monovalent cations, such as potassium ions. The reagents may be provided in the form of a solution, a concentrated solution, or powder.

In some embodiments, detection and quantification of the miRNAs includes the use of a reagent for reverse transcription of an RNA molecule. The phrase “a reagent for reverse transcription of an RNA molecule” encompasses, but is not limited to: a reverse transcriptase; an RNase inhibitor; a primer that hybridizes to a nucleic acid sequence (such as RNA or DNA); a primer that hybridizes to an adenosine oligonucleotide; and a buffer solution that provides a suitable chemical environment for optimum activity, binding kinetics, and stability of the reverse transcriptase. The reagents may be provided in the form of a solution, a concentrated solution, or powder.

In some embodiments, detection and quantification of the miRNAs is preformed using next-generation sequencing. As used herein, the term “next-generation sequencing” refers to high-throughput parallel sequencing of short fragments of single-stranded nucleic acids attached to slides or beads, such as techniques by ILLUMINA, ROCHE (454 sequencing), or ION TORRENT, THERMOFISHER. The incorporation of individual nucleotides onto single-stranded nucleic acids may be detected optically (via fluorescence of incorporated nucleotides) or by detection of hydrogen ions released during nucleotide incorporation (e.g., ion semiconductor sequencing).

In some embodiments, detection and quantification of the miRNAs is preformed using microarray detection. As used herein, the term “microarray detection” refers to methods of detecting target nucleic acids using single-stranded nucleic acid probes attached to discrete areas of a solid surface (e.g., spots on a slide or beads in microwells). Hybridization of the probes to specific nucleic acids may be detected by a variety of methods, such as using optical detection (e.g., fluorophores, chemiluminescent molecules) or radiographic detection.

In some embodiments, detection and quantification of the miRNAs includes the use of a non-natural label. As used herein, the term “non-natural label” encompasses, without limitation, one or more labeling molecules that may be bound, attached to, or associated with a biological molecule (such as a nucleic acid, nucleotide, protein, peptide, amino acid, carbohydrate, lipid, primary/secondary metabolites, or chemical product produced by a living organism) to allow detection of the molecule when associated with the biological molecule; non-natural labels are not normally associated with the biological molecule. Exemplary non-natural labels include, without limitation: antigenic tags (e.g., digoxigenin); radioisotopes (e.g., ³²P); enzymes catalyzing chemiluminescent or colorimetric chemical reactions (e.g., horseradish peroxidase or alkaline phosphatase); nucleic acid dyes (e.g., Hoechst 33342, DAPI, ethidium bromide); organic fluorophores (e.g., 6-carboxyfluorescein, tetrachlorofluorescein, fluroscein, rhodamine, or cyanine); fluorophore quenchers (e.g., tetramethylrhodamine, dimethylaminoazobenzenesulfonic acid, BLACK HOLE QUENCHERS, or IOWA BLACK dyes); protein fluorophores (e.g., green fluorescent protein); donor and acceptor fluorophores for fluorescence resonance energy transfer (e.g., fluorescein and tetramethylrhodamine, or NowGFP and mOrange); quantum dot fluorophores (e.g., metal chalcogenides, core shell semiconducting nanocrystals, or alloyed semiconductor quantum dots); and immune system-based molecules bound, attached to, or associated with non-natural labels described herein (e.g., antibodies or antibody fragments labeled with a fluorophore or catalytic enzyme).

### Statistical Models for Diagnosis

The present disclosure provides compositions and methods for the diagnosis and treatment of cancer. In an exemplary embodiment, Applicant has provided a diagnostic test for cancer (e.g., pancreatic cancer) that is both sensitive and specific. This diagnostic test relies on the use of a statistical model. In exemplary embodiments, one or more, or various combinations of, the miRNAs of Tables 1 and 2 will be quantified using the methods disclosed herein and then further analyzed using a statistical model in order to determine a diagnosis. As used herein, the term “diagnose” refers to identifying or recognizing that an individual may have a particular disease, such as cancer (e.g., pancreatic cancer).

In exemplary embodiments, the diagnostic methods of the present disclosure are performed with the use of a statistical model. As used herein, the term “statistical model” refers to a mathematical representation of observed data. The statistical model used in the methods disclosed herein can be any statistical model known in the art. Exemplary statistical models comprise one or more models selected from the group consisting of linear discriminant analysis, logistic regression, multivariate adaptive regression splines, naive Bayes, artificial neural network, support vector machine, decision tree, K nearest neighbors classifier, functional tree, least absolute deviation (LAD) tree, Bayesian network, elastic net regression, and random forest.

Disclosed herein is a group of miRNAs that can be used individually and in groups or subsets for enhanced diagnosis of cancer, (e.g., pancreatic cancer). One example of the miRNAs useful in the diagnostic methods disclosed herein are set forth in Table 2. The test miRNAs presented in Table 2 can be used as biomarkers to predict the likelihood that an individual has cancer (e.g., pancreatic cancer). In some embodiments, the levels of 1, 2, 3, 4, 5, 6, 7, 8, 9, 10, 11, or 12 of the test miRNAs in Table 2 are determined and compared to a statistical model. In an exemplary embodiment, the levels of 7 of the test miRNAs in Table 2 are determined and compared to a statistical model. In an exemplary embodiment, the levels of the one or more test miRNAs from Table 2 will be normalized. Normalization involves the use of reference miRNAs that serve as a baseline for determining the relative quantification of the test miRNAs. In an exemplary embodiment, the test miRNAs comprise one or more of the miRNAs of Table 2 and the reference miRNAs comprise one or more of the miRNAs of Table 1. In some embodiments, 1, 2, 4, or 4 of the reference miRNAs of Table 1 are used and the normalized amount of the test miRNAs is compared to a statistical model. In an exemplary embodiment, 4 of the reference miRNAs of Table 1 are used and the normalized amount of the test miRNAs is compared to a statistical model.

In an exemplary embodiment, provided herein is a method for diagnosing pancreatic in a subject comprising the steps of (a) obtaining a sample collected from the subject, (b) detecting and quantifying one or more test microRNAs selected from the group consisting of hsa-miR-192-5p, hsa-miR-98-5p, hsa-let-7g-5p, hsa-let-7f-5p, hsa-let-7a-5p, hsa-miR-122-5p, hsa-let-7d-5p, hsa-miR-340-5p, hsa-miR-194-5p, hsa-miR-323a-5p, hsa-miR-190a-3p, and hsa-miR-26b-5p in the sample, and (c) comparing the amounts of the test microRNAs determined in step (b) to a statistical model, thereby diagnosing pancreatic cancer in the subject.

In an exemplary embodiment, the statistical model used is a logistic regression. As used herein, the term “logistic regression” is a statistical model used to determine if an independent variable has an effect on a binary dependent variable. Logistic regression is used to describe data and to explain the relationship between one dependent binary variable and one or more nominal, ordinal, interval or ratio-level independent variables.

In an exemplary embodiment, the statistical model used is a neural network. As used herein, the term “artificial neural network” or “neural network” refers to a forecasting model based on a linked collection of neural units in silico that loosely model a simple mathematical model of the brain. Artificial neural networks allow identification of complex nonlinear relationships between its response variable and its predictor variables. An artificial neural network may have one or more hidden layers that each include one or more neurons that interact to produce a prediction given two or more variables.

In some embodiments, the amount of miRNAs are compared to a statistical model to calculate the probability that a subject has cancer (e.g., pancreatic cancer). In an exemplary embodiment, a neural network is used to calculate the probability that a subject has cancer (e.g., pancreatic cancer). Disclosed herein is an analytical method for diagnosing cancer (e.g., pancreatic cancer) wherein a subject is assigned as probable to have cancer based a calculation of the probability that a subject has cancer (e.g, pancreatic cancer) in a statistical model. As used herein, “probable to have cancer” means that the subject is more likely to have cancer than the statistical occurrence of cancer in the general population. In some embodiments, a subject is assigned as probable to have cancer if the probability that the subject has cancer is calculated to be at least about 5%, about 10%, about 15%, about 20%, about 25%, about 30%, 35%, about 40%, about 45%, about 50%, about 55%, about 60%, about 65%, about 70%, about 75%, about 80%, about 85%, about 90%, about 95%, or about 100%. As used herein, “probable to have pancreatic cancer” means that the subject is more likely to have pancreatic cancer than the statistical occurrence of pancreatic cancer in the general population. In some embodiments, a subject is assigned as probable to have pancreatic cancer if the probability that the subject has pancreatic cancer is calculated to be at least about 5%, about 10%, about 15%, about 20%, about 25%, about 30%, 35%, about 40%, about 45%, about 50%, about 55%, about 60%, about 65%, about 70%, about 75%, about 80%, about 85%, about 90%, about 95%, or about 100%. In an exemplary embodiment a subject is assigned as probable to have pancreatic cancer if the probability that the subject has pancreatic cancer is calculated to be at least about 50%.

The methods disclosed herein are able to determine from a sample from a subject if the subject has cancer with a high degree of accuracy. In some embodiments, the accuracy of diagnosis of cancer is at least about 50%, 55%, 60%, 65%, 70%, 75%, 80%, 85%, 90%, 95%, or 100%. In some embodiments, the accuracy of diagnosis of pancreatic cancer is at least about 50%, 55%, 60%, 65%, 70%, 75%, 80%, 85%, 90%, 95%, or 100%. In an exemplary embodiment, the accuracy of diagnosis of pancreatic cancer is at least about 80%.

The methods disclosed herein are able to determine from a sample from a subject if the subject has cancer with a high degree of specificity. In other words, the methods of the present disclosure are able to detect cancer versus common differential diagnoses with a high degree of accuracy. In some embodiments, the specificity of diagnosis of cancer is at least about 50%, 55%, 60%, 65%, 70%, 75%, 80%, 85%, 90%, 95%, or 100%. In some embodiments, the specificity of diagnosis of pancreatic cancer is at least about 50%, 55%, 60%, 65%, 70%, 75%, 80%, 85%, 90%, 95%, or 100%. In an exemplary embodiment, the specificity of diagnosis of pancreatic cancer is at least 80%.

The methods disclosed herein are able to determine from a sample from a subject if the subject has cancer with a high degree of sensitivity. In some embodiments, the sensitivity of diagnosis of cancer is at least about 50%, 55%, 60%, 65%, 70%, 75%, 80%, 85%, 90%, 95%, or 100%. In some embodiments, the sensitivity of diagnosis of pancreatic cancer is at least about 50%, 55%, 60%, 65%, 70%, 75%, 80%, 85%, 90%, 95%, or 100%. In an exemplary embodiment, the sensitivity of diagnosis of pancreatic cancer is at least 80%.

In an exemplary embodiment, provided herein is an analytical method for diagnosing pancreatic cancer in a subject, comprising the steps of: (a) detecting and quantifying one or more test microRNAs selected from the group consisting of hsa-miR-192-5p, hsa-miR-98-5p, hsa-let-7g-5p, hsa-let-7f-5p, hsa-let-7a-5p, hsa-miR-122-5p, hsa-let-7d-5p, hsa-miR-340-5p, hsa-miR-194-5p, hsa-miR-323a-5p, hsa-miR-190a-3p, and hsa-miR-26b-5p in the sample from said subject, (b) analyzing the amount of the one or more test microRNAs quantified in step a) in a neural network to determine the probability that the subject has pancreatic cancer, and (c) assigning the subject as probable to have pancreatic cancer based on the analysis of step b).

### Diagnosis and Treatment of Cancer

The methods disclosed herein can be used alone or in combination with other methods for the diagnosis and treatment of cancer.

Cancers that can be diagnosed using the methods disclosed herein include, without limitation, a solid tumor, a hematological cancer (e.g., leukemia, lymphoma, myeloma, e.g., multiple myeloma), and a metastatic lesion. In one embodiment, the cancer is a solid tumor. Examples of solid tumors include malignancies, e.g., sarcomas and carcinomas, e.g., adenocarcinomas of the various organ systems, such as those affecting the lung, breast, ovarian, lymphoid, gastrointestinal (e.g., colon), anal, genitals and genitourinary tract (e.g., renal, urothelial, bladder cells, prostate), pharynx, CNS (e.g., brain, neural or glial cells), head and neck, skin (e.g., melanoma), and pancreas, as well as adenocarcinomas which include malignancies such as colon cancers, rectal cancer, renal-cell carcinoma, liver cancer, lung cancer (e.g., non-small cell lung cancer or small cell lung cancer), cancer of the small intestine and cancer of the esophagus. The cancer may be at an early, intermediate, late stage or metastatic cancer.

In an exemplary embodiment, the cancer is pancreatic cancer. The term “pancreatic cancer,” as used herein, refers to a group of malignancies affecting the pancreas. Adenocarcinoma of the pancreas is the most common type of pancreatic cancer and starts with cancer growth in the exocrine cells. Pancreatic neuroendocrine tumors, or islet cell tumors, which start in the endocrine cells, are less common. About 95% of cancers of the exocrine pancreas are adenocarcinomas which usually start in the ducts of the pancreas (About Pancreatic Cancer, The American Cancer Society: Available at https://www.cancer.org/cancer/pancreatic-cancer/about/what-is-pancreatic-cancer.html). Less commonly, cancer develops from the cells that make the pancreatic enzymes and is known as acinar cell carcinoma. Other less common types of exocrine cancer include adenosquamous carcinomas, squamous cell carcinomas, signet ring cell carcinomas, undifferentiated carcinomas, and undifferentiated carcinomas with giant cells. (Id.)

All pancreatic cancers are classified according to the TNM staging system which is based on the evaluation of the primary tumor, lymph node status, and the presence of metastatic disease (Ansari et al. Pancreatic Cancer: Yesterday, Today, and Tomorrow, Future Oncology 2016 August; 12(16):1929-46). The TNM staging system categories pancreatic cancers in Stages 0, IA, IB, IIA, IIB, III, and IV. Stage 0 indicates that the primary tumor is carcinoma in situ, there is no regional lymph node metastasis, and no distant metastasis. Stage IA indicates that the primary tumor is limited to the pancreas and is less than or equal to 2 cm (T1), there is no regional lymph node metastasis (N0), and no distant metastasis (M0). Stage IB indicates that the primary tumor is limited to the pancreas and is greater than 2 cm (T2), there is no regional lymph node metastasis (N0), and no distant metastasis (M0). Stage IIA indicates that the primary tumor extends beyond the pancreas but without involvement of the celiac axis or the superior mesenteric artery (T3), there is no regional lymph node metastasis (N0), and no distant metastasis (M0). Stage IIB indicates any level primary tumor progression (T1-T3), regional lymph node metastasis (N1), and no distant metastasis (M0). Stage III indicates primary tumor that involves the celiac axis or the superior mesenteric artery (unresectable primary tumor) (T4), any level of lymph node involvement (N0-N1), and no distant metastasis (M0). Stage IV indicates any level of primary tumor (T0-T4), any level of lymph node involvement (N0-N1), and distant metastasis (M1).

As used herein, “at a higher risk of developing pancreatic cancer” refers to a subject who is predisposed to or statistically more likely than the general population to develop pancreatic cancer due to factors that can include genetics, age, comorbidities, etc. In some embodiments, an individual is at a higher risk for developing pancreatic cancer because they have diabetes. The term “diabetes,” as used herein, refers to a chronic, metabolic disease characterized by elevated levels of blood glucose. Diabetes can be classified as either Type 1 or Type 2. Type 1 diabetes, or insulin-dependent diabetes, is a chronic condition in which the pancreas produces little or no insulin by itself. Type 2 diabetes occurs when the body becomes resistant to insulin or doesn't make enough insulin. In some embodiments, an individual is at a higher risk for developing pancreatic cancer because they have pancreatitis. In other embodiments, an individual is at a higher risk for developing pancreatic cancer because of a family history of pancreatic cancer or pancreatitis.

In exemplary embodiments, an individual is at a higher risk for developing pancreatic cancer due to a genetic mutation. As used herein, “at a higher risk for developing pancreatic cancer due to a genetic mutation” refers to an individual that has a DNA mutation that makes the development of pancreatic cancer statistically more likely than the general population. These genetic mutations can include any mutations known in the art to be correlated with cancer or pancreatic cancer specifically. Exemplary genes in which mutations are associated with pancreatic cancer include BRCA1, BRCA2, PALB2, TP53, MLH1, CDKN2A, and ATM.

In exemplary embodiments, the methods disclosed herein are used to select a subject for more invasive testing to confirm a cancer diagnosis (e.g., pancreatic cancer). As used herein, the term “more invasive testing” refers to any type of test beyond detecting the levels of miRNA as described herein. In some embodiments, “more invasive testing” can include tests wherein a subject's sample is collected and analyzed to detect cancer, such as a biopsy or a blood draw. In some embodiments, “more invasive testing” includes an endoscopy or other exploratory procedure to detect cancer. In some embodiments, “more invasive testing” includes imaging to detect cancer. Exemplary imaging techniques include magnetic resonance imaging (MRI), computed tomography (CT) scan, x-ray, positron emission tomography and computed tomography (PET-CT) scan, ultrasound, endoscopy, and nuclear scan.

In exemplary embodiments, the methods disclosed herein are used to select a subject for surveillance of cancer to monitor if the subject develops cancer (e.g., pancreatic cancer). As used herein, the term “surveillance of cancer” can include the use of any of the “more invasive testing” methods described above, repeated on a periodic basis. In some embodiments the testing above is repeated in the following time frames: about once every 1, 2, 3, 4, 5, 6, 7, 8, 9, 10, 11, 12, 13, 14, 15, 16, 17, 18, 19, 20, 21, 22, 23, or24 weeks. In some embodiments the testing above is repeated in the following time frames: about once every 1, 2, 3, 4, 5, 6, 7, 8, 9, 10, 11, 12, 13, 14, 15, 16, 17, 18, 19, 20, 21, 22, 23, or 24 months.

In exemplary embodiments, the methods disclosed herein are used to select a subject for treatment for cancer (e.g., pancreatic cancer). As used herein, the term “treating” or “treatment” refers to relieving, reducing, or alleviating at least one symptom in a subject or effecting a delay of progression of a disease. For example, treatment can be the diminishment of one or several symptoms of a disorder or complete eradication of a disorder, such as cancer. Within the meaning of the present disclosure, the term “treat” also denotes to arrest and/or reduce the risk of worsening a disease, or prevention of at least one symptom associated with or caused by the state, disease or disorder being prevented. For example, treatments may relieve, reduce or alleviate at least one symptom of cancer (e.g., pancreatic cancer).

Surgery is an exemplary treatment for cancer (e.g., pancreatic cancer). A surgery for cancer located in the head of the pancreas is called a Whipple procedure (pancreaticoduodenectomy). A surgery called a pancreatectomy can be done to remove the left side of the pancreas. Another treatment for pancreatic cancer is a surgery to remove the entire pancreas, called total pancreatectomy.

Chemotherapy is an exemplary treatment for cancer (e.g., pancreatic cancer). A variety of exemplary chemotherapy agents can be used to treat cancer (e.g., pancreatic cancer). Taxanes can also be used to treat pancreatic cancer, such as Paclitaxel (Taxol®), Docetaxel (Taxotere®), and Albumin-bound Paclitaxel (Abraxane®). Antimetabolite drugs can be used to treat pancreatic cancer, such as Gemcitabine Hydrocholoride (Gemzar® or Infugem®), 5-fluorouracil (5-FU or Adrucil®), or Capecitabine (Xeloda®). Platinum chemotherapy can be used to treat pancreatic cancer, such as Oxaliplatin (Eloxatin®). Alkylating agents can be used to treat pancreatic cancer, such as Cisplatin (PLATINOL®). Agents that inhibit DNA replication can also be used to treat pancreatic cancer such as Irinotecan (Camptosar®) and Liposomal Irinotecan (Onivyde®). PARP inhibitors can also be used to treat pancreatic cancer, such as Olaparib (Lynparza®). Antineoplastic chemotherapy drugs can also be used to treat pancreatic cancer, such as Everolimus (Afinitor®), Erlotinib Hydrochloride (Tarceva®), Sunitinib (Sutent®) or Mitomycin.

Drug combinations are an exemplary treatment for cancer (e.g., pancreatic cancer). An exemplary drug combination is FOLFIRINOX (Folinic Acid, Fluorouracil, Irinotecan Hydrochloride, and Oxaliplatin). A further exemplary drug combination is GEMCITABINE-CISPLATIN (Gemcitabine Hydrochloride and Cisplatin). Another exemplary drug combination is GEMCITABINE-OXALIPLATIN (Gemcitabine Hydrochloride and Oxaliplatin). Still another exemplary drug combination is OFF (Oxaliplatin, Fluorouracil, and Folinic Acid).

Radiation therapy is an exemplary treatment for cancer (e.g., pancreatic cancer). Radiation therapy utilizes high-energy beams, such as those from X-rays or protons to destroy cancer cells. The radiation therapy used can be external beam radiation wherein an external beam of radiation comes from a machine and aims the radiation at the cancer in the patient. Alternatively, in internal radiation therapy, a source of radiation, such as a solid or liquid in put inside the patient's body. Chemotherapy and radiation can be combined and this is called chemoradiation.

Immunotherapy is an exemplary treatment for cancer (e.g., pancreatic cancer). Immunomodulators, such as Pembrolizumab (Keytruda®) are a type of immunotherapy that can be used to treat pancreatic cancer.

In an exemplary embodiment, provided herein is a method for treating a subject suspected of having a pancreatic cancer, the method comprising: (a) obtaining a sample collected from the subject, (b) detecting and quantifying one or more test microRNAs selected from the group consisting of hsa-miR-192-5p, hsa-miR-98-5p, hsa-let-7g-5p, hsa-let-7f-5p, hsa-let-7a-5p, hsa-miR-122-5p, hsa-let-7d-5p, hsa-miR-340-5p, hsa-miR-194-5p, hsa-miR-323a-5p, hsa-miR-190a-3p, and hsa-miR-26b-5p in the sample, (c) comparing the amounts of the test microRNAs determined in step (b) to a statistical model, and (d) selecting a subject for more invasive testing and/or surveillance of pancreatic cancer based on the comparison of step (c); and optionally administering treatment to the subject for pancreatic cancer.

In some embodiments, the treatment is a combination of the various treatment methods described herein. An exemplary treatment method is the combination of chemotherapy and immunotherapy. Another exemplary treatment method is the combination of chemotherapy and radiation. Still another exemplary treatment method is the combination of immunotherapy and radiation. Further exemplary treatment methods include the use of any of the treatment methods disclosed herein in combination with surgery (e.g., surgery and chemotherapy, surgery and immunotherapy, surgery and radiation, etc.)

### Kits

Kits are also provided for carrying out the diagnostic and treatment methods disclosed herein. The kits may optionally further comprise instructions on how to use the various components of the kits.

In an exemplary embodiment, a kit comprises at least one test probe capable of specifically hybridizing to a microRNA selected from the group consisting of hsa-miR-192-5p, hsa-miR-98-5p, hsa-let-7g-5p, hsa-let-7f-5p, hsa-let-7a-5p, hsa-miR-122-5p, hsa-let-7d-5p, hsa-miR-340-5p, hsa-miR-194-5p, hsa-miR-323a-5p, hsa-miR-190a-3p, and hsa-miR-26b-5p, or a cDNA thereof.

Kits of the invention may comprise a carrier being compartmentalized to receive in close confinement one or more containers, such as vials, test tubes, ampules, bottles and the like. Each of such containers comprise components or a mixture of components as described herein (primers, test probes, normalizing probes, test or normalizing probes with a non-natural label (e.g., a fluorescent label such as TaqMan®, Scorpions®, and LightCycler®), fluorescent dyes (e.g., SYBR® Green), solvents or buffers, reagents for amplification of a DNA sequence, reagents for reverse transcription of an RNA molecule, etc.) In general kits may also contain one or more buffers, control samples, etc.

In certain embodiments, the kit comprises one or more containers containing the test probes disclosed herein. In some embodiments, the test probes contain a detectable label. In some embodiments, the kit comprises one or more containers containing normalizing probes. In some embodiments, the normalizing probes contain a detectable label.

In exemplary embodiments, the kit comprises one or more containers containing reagents for reverse transcription of an miRNA molecule. In some embodiments, the kit comprises one or more containers containing reverse transcriptase enzyme. In some embodiments, the kit comprises one or more containers containing oligo-dT primers. In some embodiments, the kit comprises one or more containers containing dNTPs. In some embodiments, the kit comprises one or more containers containing RNase Inhibitor. In some embodiments, the kit comprises one or more containers containing a primer that hybridizes to RNA. In some embodiments, the kit comprises one or more containers containing a buffer solution that provides a suitable chemical environment for reverse transcriptase enzyme. In some embodiments, all the reagents for reverse transcription of an miRNA molecule are contained within a single container.

In exemplary embodiments, the kit comprises one or more containers containing reagents for qPCR. In some embodiments, the kit comprises one or more containers containing DNA polymerases. In some embodiments, the kit comprises one or more containers containing DNA binding dyes. In some embodiments, the kit comprises one or more containers containing probes containing a non-natural label. In some embodiments, the kit comprises one or more containers containing dNTPs.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following examples, which are included for purposes of illustration only and are not intended to be limiting.

As used herein, the terms “comprising,” “including,” “having,” and grammatical variants thereof are to be taken as specifying the stated features, integers, steps or components but do not preclude the addition of one or more additional features, integers, steps, components or groups thereof. These terms encompass the terms “consisting of” and “consisting essentially of.”

### EXAMPLES

### Example 1: Neural Networks to Produce a Diagnostic Circulating miRNA Signature from Human Sera

Summary: This example describes the development of a diagnostic test for pancreatic cancer detection that relies on miRNA expression and an advanced AI-based algorithm that calculates the probability of the disease through an artificial neural network. The method comprises a set of 10 miRNAs that may be measured using miRNA-sequencing or quantitative PCR (qPCR). For both methods an appropriately weighted algorithm was prepared that uses an input of miRNA expression data and provides the user with a probability of the sample originating from a patient with pancreatic cancer. The model was developed from 182 samples from Boston and Poland using miRNA-seq and validated on retested Poland samples and an additional 150 samples from Poland. Samples from healthy patients and patients with pancreatitis, the most common differential diagnosis for pancreatic cancer, were used to evaluate the test performance. In both instances the samples were divided randomly into sets used to train the classification models, with 20% of the samples being held out as an independent validation set to evaluate the performance of the models. The results for the miRNA-seq-based test showed a sensitivity of about 71% and a specificity of about 91%. The qPCR-based neural network slightly improved on this performance with a sensitivity of about 76% and a specificity of about 92%. These diagnostic values make the test suitable for repeated testing of patients with a high risk of pancreatic cancer, such as patients diagnosed with diabetes.

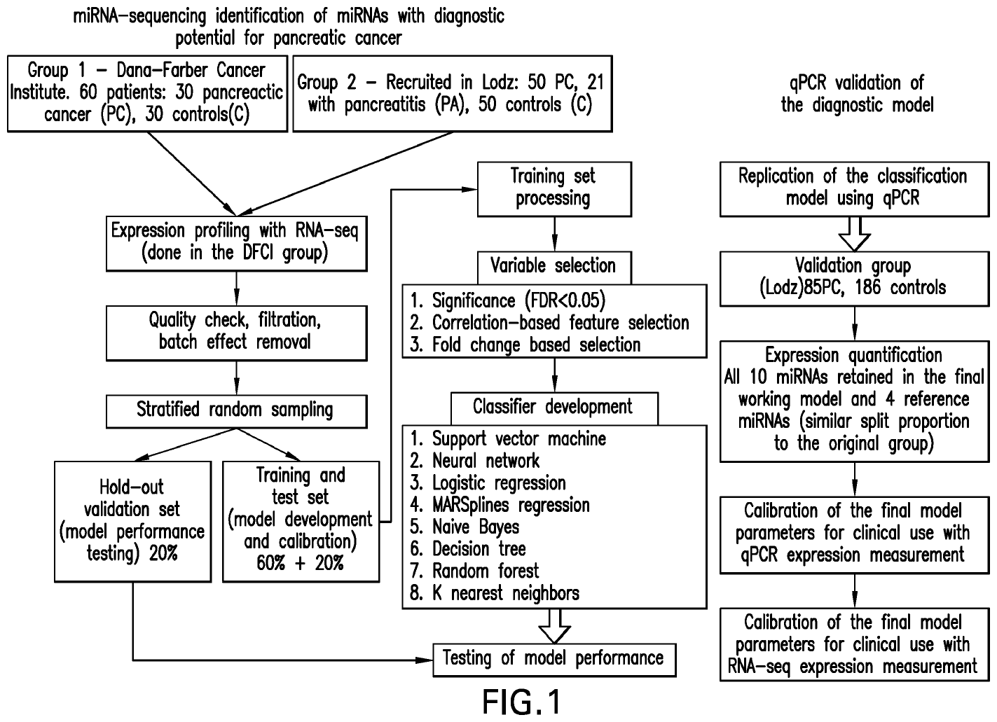

In order to produce a diagnostic circulating miRNA signature from human sera, a study population of pre-treatment subjects with 182 patients from two independent cohorts was assembled. As shown in FIG. 1, one cohort was in the US at the Dana-Farber Cancer Institute (DFCI) and the second cohort was in Poland at the Medical University of Lodz. Patients in the DFCI cohort had advanced stage pancreatic cancer (n=30) and were matched for age and sex with 30 healthy controls. Patients from Poland had pancreatic cancer (n=44: early stage 8, advanced 27, unknown 9), pancreatitis (n=28), or were clinically healthy (n=50). Small sequencing (miRNA-seq) was used to detect all known and predicted miRNAs as described earlier in ovarian cancer (Elias et al. Diagnostic Potential for a Serum miRNA Neural Network for Detection of Ovarian Cancer, Elife. 2017; 6:e28932. Published 2017 Oct. 31. doi:10.7554/eLife.28932) or radiation exposure (Fendler et al. Evolutionarily conserved serum microRNAs predict radiation-induced fatality in nonhuman primates. Sci Transl Med. 2017; 9 (379):eaal2408. doi:10.1126/scitranslmed.aal2408). As shown in FIG. 1, the patients were subsequently randomly assigned to three groups: 1) a training set for variable selection and model development, 2) a test set for calibration of a diagnostic cut-off of classification models, and 3) a validation set for performance testing of the diagnostic models on new data. Next, a series of statistical tools, including machine-learning approaches were deployed to analyze the miRNA-seq data to create an algorithm with the best performance for discriminating pancreatic cancer patients from patients with pancreatitis and healthy controls. Once the final set of miRNAs was established and the model calibration performed, we analyzed them on the validation subgroup. Despite the limited number of samples available, performance of the neural network analysis exceeded 85% accuracy. Of note, both early and advanced state cancers were identified with similar performance using the neural network approach.

The four reference miRNAs shown in Table 1 were identified for qPCR validation purposes.

TABLE 1
(Reproduced from Above):
Reference miRNAs for Normalizing Results

| SEQ | MicroRNA | |
|---|---|---|
| ID NO: | miRBase ID | Sequence |
| 1 | hsa-miR-17-5p | CAAAGUGCUUACAGUGCAGGUAG |
| 2 | hsa-miR-199a-3p | CCCAGUGUUCAGACUACCUGUUC |
| 3 | hsa-miR-28-3p | CACUAGAUUGUGAGCUCCUGGA |
| 4 | hsa-miR-92a-3p | UAUUGCACUUGUCCCGGCCUGU |

The ten miRNAs shown in Table 2 were selected to be used in the miRNA signature for diagnosing pancreatic cancer.

TABLE 2
(Reproduced from Above):
Test miRNAs for Diagnosing Pancreatic Cancer

| SEQ | MicroRNA | |
|---|---|---|
| ID NO: | miRBase ID | Sequence |
| 5 | hsa-miR-192-5p | CUGACCUAUGAAUUGACAGCC |
| 6 | hsa-miR-98-5p | UGAGGUAGUAAGUUGUAUUGUU |
| 7 | hsa-let-7g-5p | UGAGGUAGUAGUUUGUACAGUU |
| 8 | hsa-let-7f-5p | UGAGGUAGUAGAUUGUAUAGUU |
| 9 | hsa-let-7a-5p | UGAGGUAGUAGGUUGUAUAGUU |
| 10 | hsa-miR-122-5p | UGGAGUGUGACAAUGGUGUUUG |
| 11 | hsa-let-7d-5p | AGAGGUAGUAGGUUGCAUAGUU |
| 12 | hsa-miR-340-5p | UUAUAAAGCAAUGAGACUGAUU |
| 13 | hsa-miR-194-5p | UGUAACAGCAACUCCAUGUGGA |
| 14 | hsa-miR-26b-5p | UUCAAGUAAUUCAGGAUAGGU |

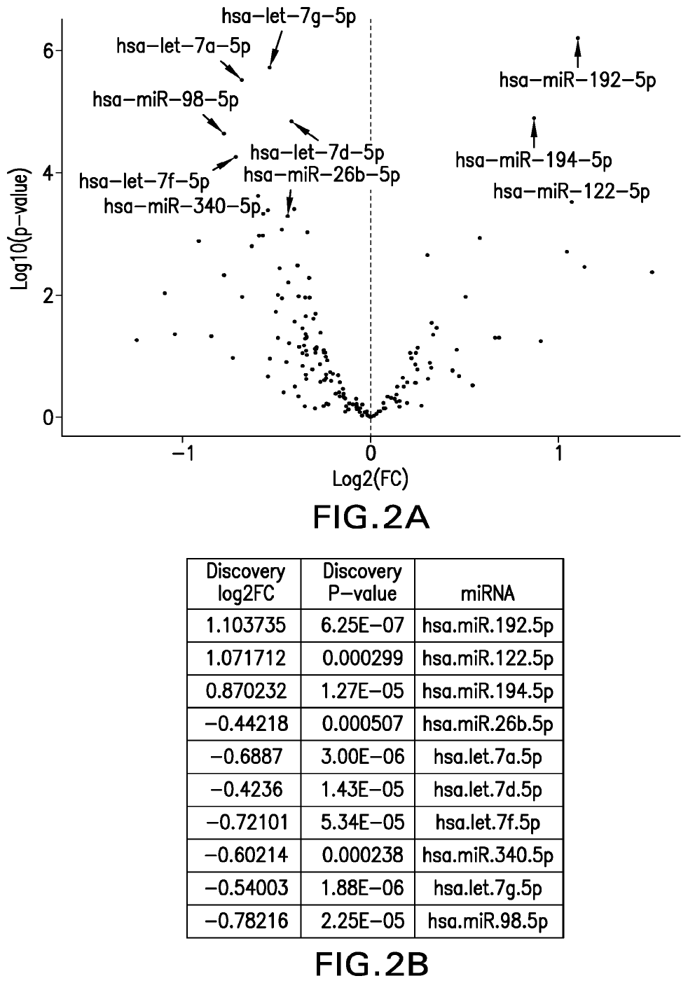

FIGS. 2A-B illustrate the variable selection process for the training set mentioned above. Ten miRNAs were selected as having a family-wise error rate (FWER) p value <0.05 (with a Bonferroni-adjusted p value). The volcano plot and table of results illustrates that for these 10 miRNAs, 3 were upregulated and 7 were downregulated.

The 10 miRNAs were used to develop a classification model using two main methods logistic regression (with backward stepwise variable selection to reduce the number of miRNAs in the analysis) and artificial neural network (with sensitivity analysis used to reduce the number of miRNAs in the analysis).

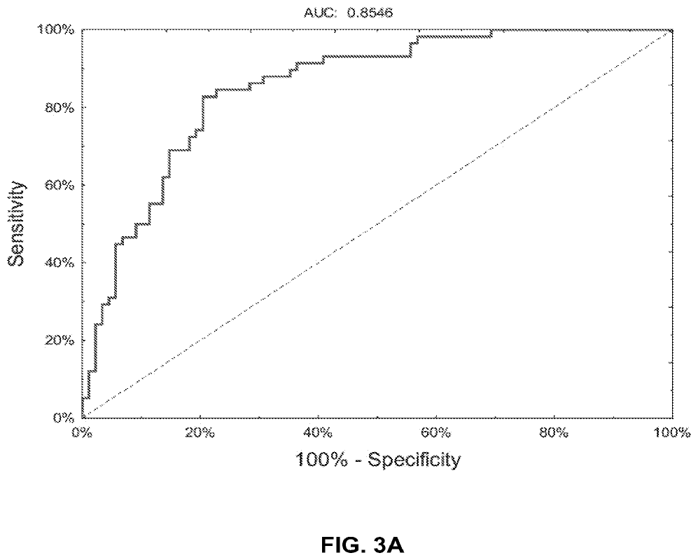

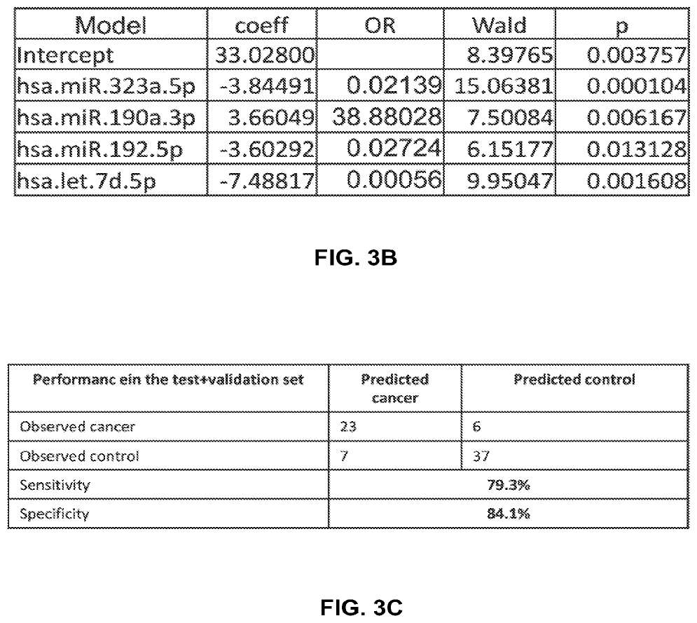

The results of the logistic regression analysis are shown in FIG. 3A-B. FIG. 3A depicts a plot of specificity versus sensitivity for the model miRNA models tested. FIG. 3B depicts the calculated values for the 4 miRNAs used in the final model. The results were calculated using a family-wise error rate (FWER) based log with a cut-off of 50%. These results show that the model fits the training and test set very well with a Hosmer Lemeshow value=4.4927 and a p value=0.810161. FIG. 3C depicts the sensitivity and specificity in detecting cancer in samples versus control. The final logistic regression model of 4 miRNAs showed a sensitivity of 79.3% and a specificity of 84.1%.

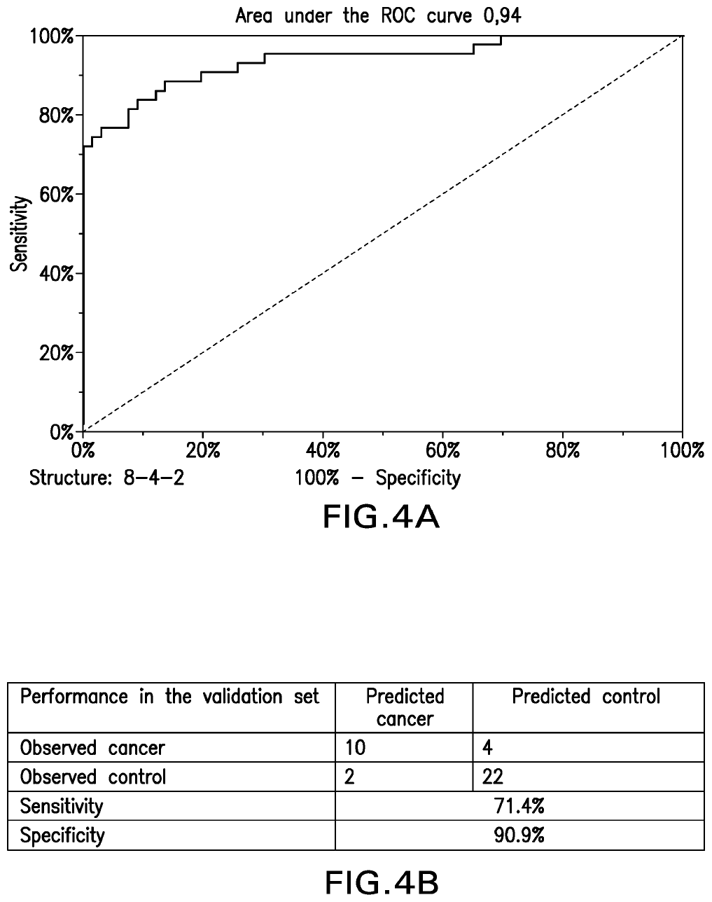

The results of the artificial neural network are shown in FIG. 4A-B. FIG. 4A depicts a plot of specificity versus sensitivity for the model miRNA models tested. The artificial neural network requires the following 8 miRNAs: hsa-miR-192-5p, hsa-let-7a-5p, hsa-let-7d-5p, hsa-miR-194-5p, hsa-miR-98-5p, hsa-let-7f-5p, hsa-miR-122-5p, and hsa-miR-340-5p. FIG. 4B depicts sensitivity and specificity in detecting cancer in samples versus control. The final artificial neural network showed a sensitivity of 71.4% and a specificity of 90.9%.

Next, as shown in FIG. 1, qPCR validation of the diagnostic model was completed. The results of the classification model were replicated using qPCR. The set of 10 miRNAs and 4 reference miRNAs was quantified in the Polish samples used in miRNA-seq and an additional 150 samples as shown in Table 3 below. QPCR based validation was performed using a custom-made array with the ten miRNAs shown in Table 2 selected as significantly differently expressed after Bonferroni correction. Next, the four miRNAs shown in Table 1 were selected as normalizers using the normiRazor tool (Grabia et al. NormiRazor: Tool Applying GPU-Accelerated Computing for Determination of Internal References in MicroRNA Transcription Studies. BMC Bioinformatics 21, 425 (2020). https://doi.org/10.1186/s12859-020-03743-8).

TABLE 3
Samples used for Replication of the Results using qPCR

| Sample Type | Polish Set 1 | Polish Set 2 |
|---|---|---|
| Healthy | 50 | 0 |
| Pancreatitis | 21 | 107 |
| Cancer | 46 | 43 |
| Atypical Cancer | 4 | 0 |
| Total | 121 | 150 |

The qPCR analysis included pre-processing of the cycle threshold (Ct) values. The three pre-processing steps for the original Ct values included 1) background-filtering (wherein values exceeding Ct values measured in blank samples were treated as non-detects), 2) imputing non-detects using an expectation-maximization (EM) algorithm, and 3) deduplication of Ct values (wherein the mean Ct value from 2 measurements was obtained). Finally, normalization (the dCt calculation) was performed using the following formula: Cq=mean of the top 3 normalizers (hsa-miR-17-5p, hsa-miR-92a-3p, and hsa-miR-199a-3p).

Next, three different techniques were used to design a final diagnostic model. These techniques included logistic regression (with backward stepwise variable selection), artificial neural network on the raw dataset, and artificial neural network using synthetic minority over-sampling technique (SMOTE) to balance the dataset. In this analysis, since it had been determined that the miRNAs are able to distinguish cancers from controls, to further the aim of creating the best possible classification tool, clinical data was also used including age and sex data to improve the performance of the final model. The data is further split into a training/test set in order to evaluate overfitting in the construction of an artificial neural network, and a validation set in order to evaluate the model performance in an unbiased way. Finally, the SMOTE oversampling technique was used to balance the set and boost performance of the classification models.

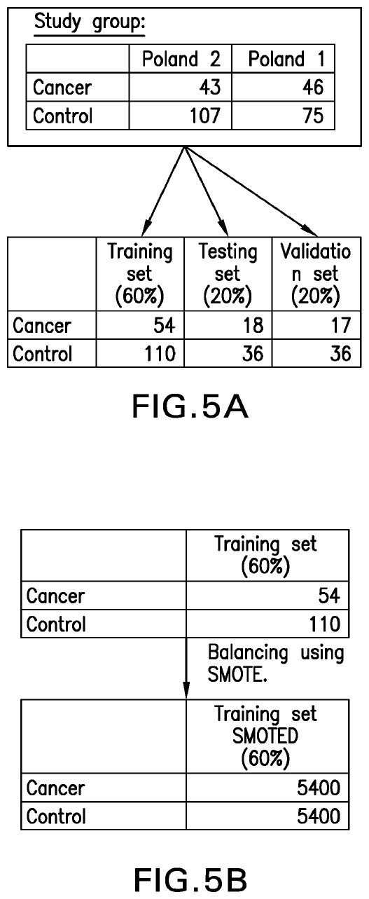

As shown in FIG. 5A, the dataset of the two sets of Polish samples was split for modeling. For the development of predictive models, the dataset was split into training, testing, and validation groups. As shown in FIG. 5B, in order to counteract the imbalance problem, a balanced dataset was created using the SMOTE technique for the training set, while the test and validation datasets remained the same.

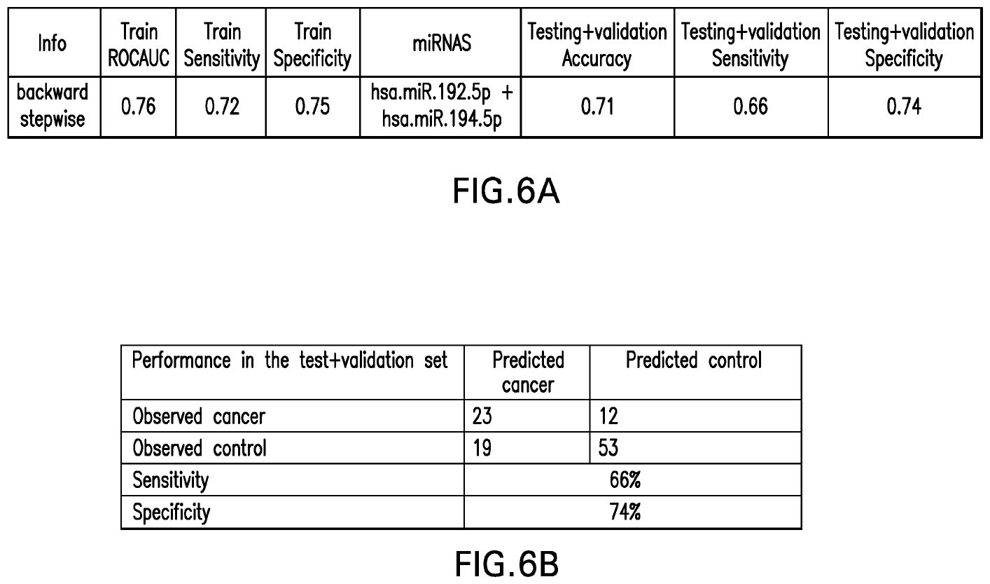

As shown in FIG. 6A-B, logistic regression of a two miRNA (hsa-miR-192-5p and hsa-miR-194-5p) model was used to evaluate the performance of both the test and validation dataset. FIG. 6B illustrates that this model has a rate of 66% sensitivity and 74% specificity in predicting cancer in samples with observed cancer versus control.

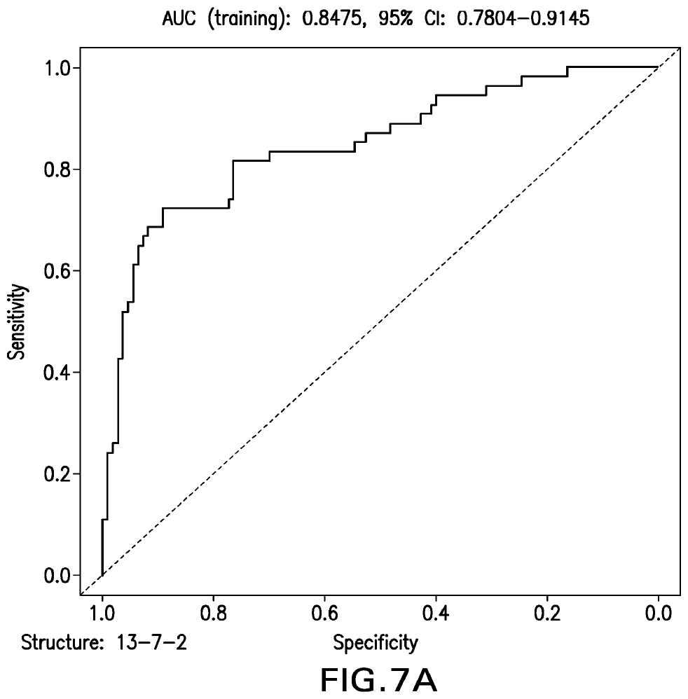

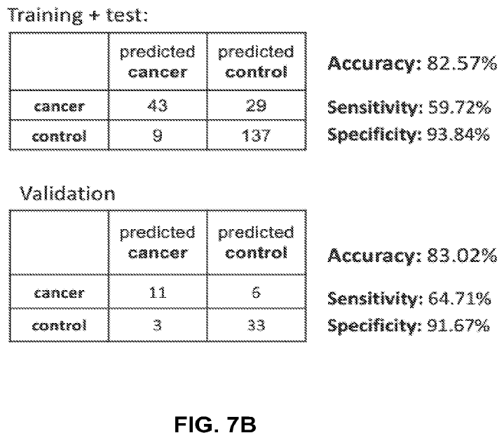

As shown in FIG. 7A-B, the neural network model of classic (non-SMOTE modified) data was tested with the clinical data including age and sex, as well as, all 10 miRNAs. The following miRNAs were used for normalization: hsa-miR-17-5p, hsa-miR-92a-3p, and hsa-miR-199a-3p. The results for this neural network model are shown in FIG. 7A-B. FIG. 7A shows the area under the ROC curve (AUC) for the training set to be 0.8475. FIG. 7B provides values of 82.57% accuracy, 59.72% sensitivity, and 93.84% specificity for the training and test datasets. FIG. 7B provides values of 83.02% for accuracy, 64.71% for sensitivity, and 91.67% for specificity for the validation data set.

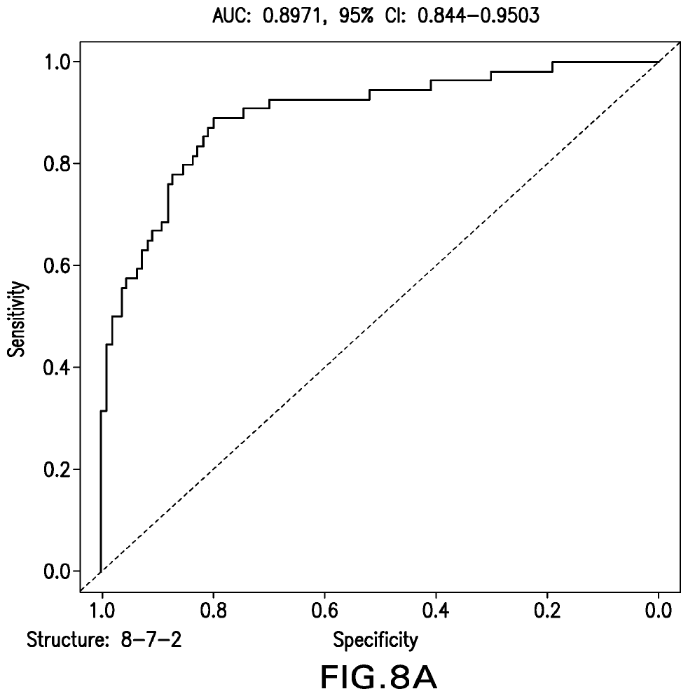

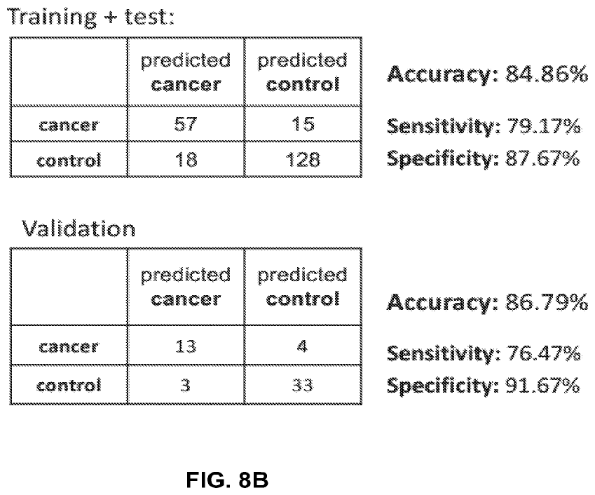

As shown in FIG. 8A-B, the neural network model on the SMOTE balanced dataset was tested using the clinical data set and a trimmed miRNA set comprising hsa-miR-192-5p, hsa-let-7a-5p, hsa-miR-194-5p, hsa-let-7f-5p, hsa-miR-122-5p, hsa-miR-340-5p, and hsa-miR-26b-5p. The following miRNAs were used for normalization: hsa-miR-17-5p, hsa-miR-92a-3p, and hsa-miR-199a-3p. FIG. 8A shows the AUC for the data set to be 0.8971. FIG. 8B provides values of 84.86% accuracy, 79.17% sensitivity, and 87.67% specificity for the training and test datasets. FIG. 8B provides values of 86.79% accuracy, 76.47% sensitivity, and 91.67% specificity for the validation dataset.

These results show that the identified set of miRNAs can be used to identify patients with pancreatic cancer and distinguish them from healthy controls or patients with pancreatitis with a sensitivity of 76-82% and a specificity of 83-91% depending on the tool of choice. The artificial networks can be used jointly or separately depending on the a priori risk of pancreatic cancer and the doctor's preference to use the miRNA-based test as a screening test or a confirmatory test.

1. A method for diagnosing pancreatic cancer in a subject, the method comprising:
(a) obtaining a sample collected from the subject;
(b) detecting and quantifying one or more test microRNAs selected from the group consisting of hsa-miR-192-5p (SEQ ID NO: 5), hsa-miR-98-5p (SEQ ID NO: 6), hsa-let-7g-5p (SEQ ID NO: 7), hsa-let-7f-5p (SEQ ID NO: 8), hsa-let-7a-5p (SEQ ID NO: 9), hsa-miR-122-5p (SEQ ID NO: 10), hsa-let-7d-5p (SEQ ID NO: 11), hsa-miR-340-5p (SEQ ID NO: 12), hsa-miR-194-5p (SEQ ID NO: 13), hsa-miR-323a-5p (SEQ ID NO: 15), hsa-miR-190a-3p (SEQ ID NO: 16), and hsa-miR-26b-5p (SEQ ID NO: 14) in the sample; and
(c) comparing the amounts of the test microRNAs determined in step (b) to a statistical model, thereby diagnosing pancreatic cancer in the subject.

2-5. (canceled)

6. The method of claim 1, wherein step (b) comprises detecting and quantifying either:
hsa-miR-192-5p (SEQ ID NO: 5), hsa-miR-98-5p (SEQ ID NO: 6), hsa-let-7f-5p (SEQ ID NO: 8), hsa-let-7a-5p (SEQ ID NO: 9), hsa-miR-122-5p (SEQ ID NO: 10), hsa-let-7d-5p (SEQ ID NO: 11), hsa-miR-340-5p (SEQ ID NO: 12), hsa-miR-194-5p (SEQ ID NO: 13), and hsa-let-7g-5p (SEQ ID NO: 7); or
hsa-miR-192-5p (SEQ ID NO: 5), hsa-miR-98-5p (SEQ ID NO: 6), hsa-let-7f-5p (SEQ ID NO: 8), hsa-let-7a-5p (SEQ ID NO: 9), hsa-miR-122-5p (SEQ ID NO: 10), hsa-let-7d-5p (SEQ ID NO: 11), hsa-miR-340-5p (SEQ ID NO: 12), hsa-miR-194-5p (SEQ ID NO: 13), and hsa-miR-26b-5p (SEQ ID NO: 14).

7. The method of claim 1, wherein step (b) comprises detecting and quantifying hsa-miR-323a-5p (SEQ ID NO: 15), hsa-miR-190a-3p (SEQ ID NO: 16), hsa-miR-192-5p (SEQ ID NO: 5), and hsa-let-7d-5p (SEQ ID NO: 11).

8. The method of claim 1, wherein step (b) comprises detecting and quantifying hsa-miR-192-5p (SEQ ID NO: 5) and hsa-miR-194-5p (SEQ ID NO: 13).

9. (canceled)

10. The method of claim 1, wherein step b) comprises detecting and quantifying at least 2, 3, 4, 5, 6, 7, 8, 9, or 10 of the test microRNAs.

11. The method of claim 1, further comprising:
(d) detecting and quantifying one or more normalizing microRNAs selected from the group consisting of hsa-miR-17-5p (SEQ ID NO: 1), hsa-miR-199a-3p (SEQ ID NO: 2), hsa-miR-28-3p (SEQ ID NO: 3), and hsa-miR-92a-3p (SEQ ID NO: 4) in the sample; and
(e) normalizing the amounts of the test microRNAs using the amounts of the normalizing microRNAs quantified in step (d).

12-13. (canceled)

14. The method of claim 11, wherein step (d) comprises detecting and quantifying at least 2 or 3 of the normalizing microRNAs.

15-22. (canceled)

23. A kit comprising at least one test probe capable of specifically hybridizing to a microRNA selected from the group consisting of hsa-miR-192-5p (SEQ ID NO: 5), hsa-miR-98-5p (SEQ ID NO: 6), hsa-let-7g-5p (SEQ ID NO: 7), hsa-let-7f-5p (SEQ ID NO: 8), hsa-let-7a-5p (SEQ ID NO: 9), hsa-miR-122-5p (SEQ ID NO: 10), hsa-let-7d-5p (SEQ ID NO: 11), hsa-miR-340-5p (SEQ ID NO: 12), hsa-miR-194-5p (SEQ ID NO: 13), hsa-miR-323a-5p (SEQ ID NO: 15), hsa-miR-190a-3p (SEQ ID NO: 16), and hsa-miR-26b-5p (SEQ ID NO: 14), or a cDNA thereof.

24-28. (canceled)

29. The kit of claim 23, comprising:
a test probe that specifically hybridizes to hsa-miR-192-5p (SEQ ID NO: 5), and
a test probe that specifically hybridizes to hsa-miR-194-5p (SEQ ID NO: 13), or a cDNA thereof.

30. (canceled)

31. The kit of claim 23, comprising at least 2, 3, 4, 5, 6, 7, 8, 9, or 10 test probes.

32. The kit of claim 31, further comprising at least one normalizing probe capable of specifically hybridizing to a microRNA selected from the group consisting of hsa-miR-17-5p (SEQ ID NO: 1), hsa-miR-199a-3p (SEQ ID NO: 2), hsa-miR-28-3p (SEQ ID NO: 3), and hsa-miR-92a-3p (SEQ ID NO: 4), or a cDNA thereof.

33-39. (canceled)

40. A method for treating a subject suspected of having a pancreatic cancer, the method comprising:
(a) obtaining a sample collected from the subject;
(b) detecting and quantifying one or more test microRNAs selected from the group consisting of hsa-miR-192-5p (SEQ ID NO: 5), hsa-miR-98-5p (SEQ ID NO: 6), hsa-let-7g-5p (SEQ ID NO: 7), hsa-let-7f-5p (SEQ ID NO: 8), hsa-let-7a-5p (SEQ ID NO: 9), hsa-miR-122-5p (SEQ ID NO: 10), hsa-let-7d-5p (SEQ ID NO: 11), hsa-miR-340-5p (SEQ ID NO: 12), hsa-miR-194-5p (SEQ ID NO: 13), hsa-miR-323a-5p (SEQ ID NO: 15), hsa-miR-190a-3p (SEQ ID NO: 16), and hsa-miR-26b-5p (SEQ ID NO: 14) in the sample;
(c) comparing the amounts of the test microRNAs determined in step (b) to a statistical model; and
(d) selecting a subject for more invasive testing and/or surveillance of pancreatic cancer based on the comparison of step (c); and optionally administering treatment to the subject for pancreatic cancer.

41-44. (canceled)

45. The method of claim 40, wherein (b) comprises detecting and quantifying either:
hsa-miR-192-5p (SEQ ID NO: 5), hsa-miR-98-5p (SEQ ID NO: 6), hsa-let-7f-5p (SEQ ID NO: 8), hsa-let-7a-5p (SEQ ID NO: 9), hsa-miR-122-5p (SEQ ID NO: 10), hsa-let-7d-5p (SEQ ID NO: 11), hsa-miR-340-5p (SEQ ID NO: 12), hsa-miR-194-5p (SEQ ID NO: 13), and hsa-let-7g-5p (SEQ ID NO: 7); or
hsa-miR-192-5p (SEQ ID NO: 5), hsa-miR-98-5p (SEQ ID NO: 6), hsa-let-7f-5p (SEQ ID NO: 8), hsa-let-7a-5p (SEQ ID NO: 9), hsa-miR-122-5p (SEQ ID NO: 10), hsa-let-7d-5p (SEQ ID NO: 11), hsa-miR-340-5p (SEQ ID NO: 12), hsa-miR-194-5p (SEQ ID NO: 13), and hsa-miR-26b-5p (SEQ ID NO: 14).

46. The method of claim 40, wherein step (b) comprises detecting and quantifying hsa-miR-323a-5p (SEQ ID NO: 15), hsa-miR-190a-3p (SEQ ID NO: 16), hsa-miR-192-5p (SEQ ID NO: 5), and hsa-let-7d-5p (SEQ ID NO: 11).

47. The method of claim 40, wherein step (b) comprises detecting and quantifying hsa-miR-192-5p (SEQ ID NO: 5) and hsa-miR-194-5p (SEQ ID NO: 13).

48. (canceled)

49. The method of claim 40, wherein step b) comprises detecting and quantifying at least 2, 3, 4, 5, 6, 7, 8, 9, or 10 of the test microRNAs.

50. The method of claim 40, further comprising:
(e) detecting and quantifying one or more normalizing microRNA selected from the group consisting of hsa-miR-17-5p (SEQ ID NO: 1), hsa-miR-199a-3p (SEQ ID NO: 2), hsa-miR-28-3p (SEQ ID NO: 3), and hsa-miR-92a-3p (SEQ ID NO: 4) in the sample; and
(f) normalizing the quantified amounts of the test microRNAs using the amounts of the normalizing microRNAs quantified in step (d).

51-52. (canceled)

53. The method of claim 50, wherein step (e) comprises detecting and quantifying at least 2 or 3 of the normalizing microRNAs.

54-82. (canceled)

83. An analytical method for diagnosing pancreatic cancer in a subject, comprising the steps of:
a) detecting and quantifying one or more test microRNAs selected from the group consisting of hsa-miR-192-5p (SEQ ID NO: 5), hsa-miR-98-5p (SEQ ID NO: 6), hsa-let-7g-5p (SEQ ID NO: 7), hsa-let-7f-5p (SEQ ID NO: 8), hsa-let-7a-5p (SEQ ID NO: 9), hsa-miR-122-5p (SEQ ID NO: 10), hsa-let-7d-5p (SEQ ID NO: 11), hsa-miR-340-5p (SEQ ID NO: 12), hsa-miR-194-5p (SEQ ID NO: 13), hsa-miR-323a-5p (SEQ ID NO: 15), hsa-miR-190a-3p (SEQ ID NO: 16), and hsa-miR-26b-5p (SEQ ID NO: 14) in the sample from said subject;
b) analyzing the amount of the one or more test microRNAs quantified in step a) in a neural network to determine the probability that the subject has pancreatic cancer; and
c) assigning the subject as probable to have pancreatic cancer based on the analysis of step b).

84-92. (canceled)

93. The method of claim 83, wherein
step (a) further comprises detecting and quantifying one or more normalizing microRNAs selected from the group consisting of hsa-miR-17-5p (SEQ ID NO: 1), hsa-miR-199a-3p (SEQ ID NO: 2), hsa-miR-28-3p (SEQ ID NO: 3), and hsa-miR-92a-3p (SEQ ID NO: 4) in the sample; and
step (b) further comprises normalizing the amounts of the test microRNAs using the amounts of the normalizing microRNAs.

94-122. (canceled)
